(19) United States
(12) Patent Application Publication
(10) Pub. No.: US 2023/0044220 A1
(43) Pub. Date: Feb. 9, 2023

(54) TREATMENT OF CHRONIC PAIN

(71) Applicant: UCL BUSINESS LTD, London (GB)

(72) Inventors: James COX, Ely (GB); John WOOD, London (GB)

(21) Appl. No.: 17/786,643

(22) PCT Filed: Dec. 18, 2020
(86) PCT No.: PCT/GB2020/053296

§ 371 Date: Jun. 17, 2022

(30) Foreign Application Priority Data
Dec. 19, 2019 (GB) 1918879.6

(51) Int. Cl. A61K 48/00; A61P 25/04; C12N 9/22; C12N 15/11

(52) U.S. Cl. CPC A61K 48/0058; A61P 25/04; C12N 9/22; C12N 15/11

(57) ABSTRACT

The present invention relates to expression constructs and viral and other vectors for the treatment and/or prevention of chronic pain.

### FIELD OF THE INVENTION

The present invention relates to expression constructs and viral and other vectors for the treatment and/or prevention of chronic pain.

### BACKGROUND OF THE INVENTION

Pain is the greatest clinical challenge of the age, affecting half the population with 7% of the population suffering debilitating chronic pain. The pharmaceutical industry has developed few effective drugs for the treatment or prevention of chronic pain. There is thus a need to develop alternative therapies to treat patients with chronic pain, such as cancer pain.

Most pain conditions require input to the central nervous system from specialised peripheral sensory neurons that drive pain perception. Silencing or muting sensory neuron activity linked to pain pathways is an attractive approach to treating pain. Amongst the most distressing of human pain conditions is bone cancer pain resulting from metastases from common cancer such as breast cancer or prostate cancer. These conditions can be modelled in mice. The present invention describes an effective gene therapy treatment for pain that has been validated in bone cancer pain in mice and its future utility in a range of human pain conditions.

Gene therapy can encompass genome editing using CRISPR methods to silence or delete genes, delivery of antisense oligonucleotides or siRNA to block protein synthesis, or the delivery of genes that regulate neuronal activity to inhibit pain pathways. AAV is a vector that is suited for human gene therapy as it is non-pathogenic.

### SUMMARY OF THE INVENTION

The present invention encompasses the use of gene therapy targeted to sensory neurons that drive pain pathways in order to mimic the analgesia found in some rare human pain-free mutants. To do this there is a need to specifically manipulate gene expression only in sensory neurons. To this end the inventors have designed and tested DNA sequences from the promoter of the sensory neuron gene Advillin. The inventors have identified a conserved Advillin promoter fragment small enough to drive genes in a selective manner in sensory neurons using an AAV viral vector suitable for human gene therapy.

Advillin is predominantly expressed in dorsal root ganglion (DRG) and trigeminal ganglion sensory neurons, and is expressed in almost all DRG sensory neurons. The predominant expression in sensory neurons makes the Advillin promoter suitable for pain gene therapy studies.

The present invention is based on the discovery of a conserved Advillin promoter region within the gene region upstream of the Advillin coding region, which allows for expression of payloads in sensory neurons. In addition, the promoter region is no more than 500 nucleotides in length. Thus, it can be operably linked to payload sequences in a viral vector, such as the adenovirus-associated vector AAV. These viral vectors can be used in gene therapy applications to express payload sequences in sensory neurons. For example, the viral vectors can be used to introduce payloads in the dorsal root or trigeminal ganglion that inhibit or regulate the expression or function of a protein involved in a pain pathway. Inhibiting or regulating the expression or function of a protein involved in a pain pathway leads to the abrogation of pain in subjects in need thereof. In addition the delivery of new genetic payloads—for example DREADD receptors driven by the promoter is a useful route to gene therapy.

Furthermore, the inventors have shown that the viral vectors of the present invention can be used to abrogate chronic pain pathways, whilst not silencing acute pain pathways in some indications.

Accordingly, the invention provides:

An expression construct comprising in a 5′ to 3′ direction:
(a) (i) an Advillin (Avil) promoter consisting of:
(1) a sequence of no more than 500 contiguous nucleotides comprising SEQ ID NO:1, or
(2) a sequence of no more than 500 contiguous nucleotides having 90% sequence identity to SEQ ID NO: 1, that retains the ability to express a payload sequence in dorsal root ganglion (DRG) neurons or the trigeminal ganglia; and
   (b) a payload sequence,
   wherein the promoter of (a) is operably linked to the payload sequence of (b).

The Invention Also Provides:

A vector or viral vector comprising the expression construct according to the invention, a host cell that produces the vector or viral vector according to the invention, and a pharmaceutical composition comprising the vector or viral vector according to the invention, and a pharmaceutically acceptable carrier.

The invention also provides methods of treatment or prevention of chronic pain in a patient thereof by administration of the vector, viral vector or pharmaceutical composition according to the invention, the use of the vector, viral vector or pharmaceutical composition according to the invention in the manufacture of a medicament for the treatment or prevention of chronic pain and the vector, viral vector or pharmaceutical composition according to the invention for use in a method of treating or preventing chronic pain.

### BRIEF DESCRIPTION OF THE FIGURES

FIG. 1: Plasmid maps generated using SnapGene for Short mouse, human, dog and pig Avil and human, mouse EFS driving TurboGFP. No promoter TurboGFP as a negative control.

FIG. 2: Avil promoter plasmids driving TurboGFP in mouse dorsal root ganglion (DRG) cultures (A-C) and CAD cells (D).

A (72 hrs, scale bar 20 μM); B (48 hrs, scale bar 100 μM); C (72 hrs, scale bar 50 μM); D (72 hrs post transfection). Hs=human promoter, Ms=mouse promoter.

FIG. 3: AAV9 plasmid maps generated using SnapGene for short mouse Avil promoter sequence driving Cre recombinase and CMV promoter sequence driving eGFP.

FIG. 4: Intrathecal injection of short mouse Avil-Cre AAV9 into CAG foxed stop tdTomato line. L5/L6 DRGs express tdTomato in the mice injected with the short mouse Avil-Cre AAV9 (tissue fixed 7 weeks post virus delivery) FIG. 5: Intraperitoneal injection of (A) short mouse Avil-Cre AAV9 and (B) CMV-eGFP AAV9 into ˜P7 pups from the CAG foxed stop tdTomato line. L5 DRGs express (A) tdTomato and (B) eGFP (tissue fixed 12 weeks post virus delivery).

FIG. 6: Intraperitoneal injection of (A) short mouse Avil-Cre AAV9 and (B) CMV-eGFP AAV9 into ˜P7 pups from the CAG foxed stop tdTomato line. The heart had a limited number of cells that were positive for tdTomato in mice injected with AAV9 short mouse Avil-Cre recombinase. In comparison, numerous heart cells expressed eGFP following injection with AAV9 CMV-eGFP. Tissue fixed 12 weeks post virus delivery.

FIG. 7: Plasmid maps generated using SnapGene for short mouse Avil promoter sequence driving (A) SaCas9 or (B) dSaCas9-KRAB in AAV1.

FIG. 8: Baseline data were obtained for three different sets of male and female mice that were injected intrathecally with (1) control virus expressing eGFP, (2) silencing virus mAvil-dSaCas9-KRAB or (3) editing virus mAvil-SaCas9 directed to Scn9a by guide RNAs. Mice were tested for their motor function on an accelerating Rotarod, and for innocuous sensation using von Frey filaments. Acute pain was measured using heat sensitivity with a Hargreaves apparatus. Mechanical pressure evoked pain was measured with a Randall Sellito apparatus. Four weeks after virus injection, the groups of mice were re-tested for motor function, innocuous sensation and response to noxious pressure and heat. There was little change to acute pain thresholds, potentially reflecting the fact that editing occurs only in a subset of all sensory neurons. This is potentially valuable in terms of treating chronic pain whilst maintaining the ability to detect and respond to dangerous insults.

FIG. 9: Limb use score in cancer-induced bone pain model for (A) males and females, (B) males and (C) females intrathecally injected six weeks previously with AAV1 viruses that excise Scn9a (circles), silence Scn9a (triangles) or eGFP controls (squares).

FIG. 10: Weight bearing in cancer-induced bone pain model for (A) males and females, (B) males and (C) females intrathecally injected six weeks previously with AAV1 viruses that excise Scn9a (circles), silence Scn9a (triangles) or eGFP controls (squares).

FIG. 11: Survival curve in cancer-induced bone pain model for (A) males and females, (B) males and (C) females intrathecally injected six weeks previously with AAV1 viruses that excise Scn9a (grey hashed line), silence Scn9a (dotted line) or eGFP controls (black solid line).

### BRIEF DESCRIPTION OF THE SEQUENCES

SEQ ID NO: 1—Advillin consensus sequence
SEQ ID NO: 2—human Advillin promoter fragment
SEQ ID NO: 3—mouse Advillin promoter fragment
SEQ ID NO: 4—pig Advillin promoter fragment
SEQ ID NO: 5—dog Advillin promoter fragment
SEQ ID NO: 6—human Advillin larger promoter region
SEQ ID NO: 7—mouse Advillin larger promoter region
SEQ ID NO: 8—pig Advillin larger promoter region
SEQ ID NO: 9—dog Advillin larger promoter region
SEQ ID NO: 10—Scn9a guide sequence 1
SEQ ID NO: 11—Scn9a guide sequence 2
SEQ ID NO: 12—Scn9a guide sequence 3

### SEQ ID NO: 13—SaCas9

SEQ ID NO:14—dSaCas9
SEQ ID NO:15—KRAB domain

### SEQ ID NO:16—NLS-SaCas9-NLS-3xHA

### SEQ ID NO:17—HA-NLS-dSaCas9-NLS-KRAB-NLS

SEQ ID NO:18—Human EFS promoter sequence
SEQ ID NO:19—Mouse EFS promoter sequence

### DETAILED DESCRIPTION OF THE INVENTION

It is to be understood that different applications of the disclosed polynucleotide sequences may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural references unless the content clearly dictates otherwise. Thus, for example, reference to “a polynucleotide” includes “polynucleotides”, reference to “a promoter” includes “promoters”, reference to “a vector” includes two or more such vectors, and the like.

The terms Advillin and Avil are used interchangeably in this specification.

All publications, patents and patent applications cited herein, whether supra or infra, are hereby incorporated by reference in their entirety.

The present invention concerns gene therapy for the prevention and treatment of chronic pain.

The present invention concerns gene therapy for the treatment and/or prevention of chronic pain in a patient in need thereof. The patient is preferably a mammal. The mammal may be a commercially farmed animal, such as a horse, a cow, a sheep or a pig, a laboratory animal, such as a mouse or a rat, or a pet, such as a cat, a dog, a rabbit or a guinea pig. The patient is more preferably human.

The expression constructs and vectors of the present invention can be used to silence the expression or activity of genes involved in chronic pain pathways, as well as deliver exogenous genes that diminish sensory neuron activity.

### Target Genes

Genes to be silenced by use of the promoter of the invention can be considered as target genes. The genes to be silenced are expressed in sensory neurons, for example the DRG and/or the trigeminal ganglia. Any gene that is involved in chronic pain pathways and is expressed in the DRG and/or trigeminal ganglia can be considered as a target gene of the invention. Exemplary target genes of the invention include those that encode voltage-gated sodium and calcium channels, for example, Nav1.7, Nav1.8, Cav2.2; other sodium, calcium and chloride channels, such as Nax and Cav2.3; TRP channels, such as TRPA1; ligand gated ion channels, such as ASIC1, 2 or 3; GPCRs, such as GalR2; HCN channels, such as HCN2; enzymes such as Cox-2; neuropeptides such as CGRP; regulatory RNAs and proteins such as PR domain zinc finger protein 12 (PRDM12) and Zinc finger homeobox 2 (ZFHX2).

In a preferred embodiment, the sodium voltage-gated ion channel Nav1.7 or Nav1.8 is silenced. In a preferred embodiment, a gene that expresses a subunit of the sodium voltage-gated ion channel Nav1.7 or Nav1.8 is silenced. In a preferred embodiment, the sodium voltage-gated ion channel Nav1.7 alpha subunit 9 gene (SCN9A) is silenced. In a preferred embodiment, the calcium voltage-gated ion channel Cav2.2 is silenced. In a preferred embodiment, a gene that expresses a subunit of the calcium voltage-gated ion channel Cav2.2 is silenced. In a preferred embodiment, a gene that expresses an alpha-1 subunit of the calcium voltage-gated ion channel Cav2.2 (CACNA1B) is silenced.

### Conditions to be Treated

The promoter of the present invention is useful in order to treat or prevent chronic pain in a patient in need thereof. In the present invention the conditions to be treated include chronic pain such as cancer pain, cancer-associated bone pain, rheumatoid arthritis and osteoarthritis, trigeminal neuralgia, headache, migraine, fibromyalgia, diabetic neuropathy and other neuropathy associated pain, neuropathic pain and idiopathic pain. In preferred embodiments the conditions to be treated include rare diseases like erythromelalgia (Dib-Hajj et al. 2019) and paroxysmal extreme pain disorder (Choi et al. 2011).

### Silencing Mechanisms

In an embodiment of the invention the promoter of the present invention is used in order to silence target gene expression and or/activity in sensory neurons, such as the DRG and/or trigeminal ganglia. These genes are associated with chronic pain pathways. A variety of mechanisms to silence gene expression or activity are encompassed by the present invention. The present invention also involves the diminishing or silencing of sensory neuron activity, such as the DRG and/or trigeminal ganglia activity.

The term silencing used herein encompasses diminishing, inhibition or downregulation of gene expression, diminishing, inhibition or downregulation of transcription, diminishing, inhibition or downregulation of translation, and/or diminishing inhibition or downregulation of protein activity. The diminishing, inhibition or downregulation can be direct, or indirect. Methods of determining the level of diminishing, inhibition or downregulation of gene expression, diminishing, inhibition or downregulation of transcription, diminishing, inhibition or downregulation of translation, and/or diminishing inhibition or downregulation of protein activity are known to the skilled person. Examples include in situ hybridisation to determine gene expression, immunoblotting to determine protein expression and electrophysiology to determine protein activity. The diminishing, inhibition or downregulation can be complete or partial. Silencing as described herein can be considered to encompass a 10% diminution, inhibition or downregulation, a 20% diminution, inhibition or downregulation, a 30% diminution, inhibition or downregulation, a 40% diminution, inhibition or downregulation, a 50% diminution, inhibition or downregulation, a 60% diminution, inhibition or downregulation, a 70% diminution, inhibition or downregulation, a 80% diminution, inhibition or downregulation, a 90% diminution, inhibition or downregulation, or a 100% diminution, inhibition or downregulation in gene expression, transcription, translation and/or protein activity.

The term silencing used herein also encompasses the diminishing, inhibition or downregulation of sensory neuron activity and/or function. The term silencing used herein also encompasses the diminishing, inhibition or downregulation of the activity and/or function of sensory neurons such as the DRG and/or trigeminal ganglia. The silencing of activity of said sensory neurons results in the inhibition of chronic pain pathways. The inhibition can be complete or partial. Inhibition of chronic pain pathways as described herein can be considered to encompass a 10% inhibition, a 20% inhibition, a 30% inhibition, a 40% inhibition, a 50% inhibition, a 60% inhibition, a 70%, inhibition, a 80% inhibition, a 90% inhibition, or a 100% inhibition of chronic pain pathways. Determining the level of inhibition of chronic pain pathways can be carried out by the skilled person using methods known in the art.

Silencing the activity and/or function of sensory neurons as described herein involves the use of the Avil promoter sequence of the invention to express a payload sequence in the sensory neuron that results in the activity and/or function of the neuron being silenced.

Determining whether the activity or function of sensory neurons, for example the DRG and/or trigeminal ganglia, is compromised can be carried out using methods known to the skilled person, for example by measurement of electrophysiological input into the spinal cord. Silencing the activity and/or function of sensory neurons, such as the DRG and/or trigeminal ganglia, can be complete or partial. Silencing the activity and/or function of sensory neurons, such as the DRG and/or trigeminal ganglia, can be considered to encompass a 10% reduction, a 20% reduction, a 30% reduction, a 40% reduction, a 50% reduction, a 60% reduction, a 70% reduction, a 80% reduction, a 90% reduction, or a 100% reduction in activity and/or function.

In all embodiments of the invention, the mechanisms of silencing described herein require the use of an Avil promoter sequence of the invention to express a payload sequence in sensory neurons such as the DRG and/or the trigeminal ganglia. In all embodiments of the invention, the mechanism of silencing requires the use of an Avil promoter sequence of the invention to express a payload sequence in the DRG and/or the trigeminal ganglia.

Exemplary payload sequences are described in more detail below.

Genes to be expressed by use of the promoter of the invention in order to diminish sensory neuron activity can be considered as payload sequences. Genes to be expressed by use of the promoter of the invention in order to diminish DRG or trigeminal ganglia activity can be considered as payload sequences. In an embodiment of the invention, the payload sequence comprises a potassium channel, such as KCNS1. In an embodiment of the invention, the payload sequence comprises a gene involved in the expression or delivery of opioid peptide genes. Examples of opioid peptide genes include proenkephalin (PENK) and prodynorphin. In an embodiment of the invention, dominant gene mutants that diminish sensory neuron activity, such as the ZFHX2 mutant p.R1913K, may be expressed as the payload sequence.

In some embodiments of the invention, the payload sequence can comprise sequences that act to silence gene expression, transcription, translation and/or protein activity. In such embodiments of the invention, the payload sequence can comprise a double-stranded RNA, a ncRNA, a shRNA, siRNA, a miRNA, a CRISPR enzyme sequence such as Cas-9, dCas-9, SaCas-9, dSaCas-9, dSaCas-9-KRAB, a guide RNA, zinc-finger proteins (ZFPs), transcription activator-like effector nucleases (TALENs) and/or DREADDs.

### Genes to be Delivered

In some embodiments of the invention, the payload sequence can comprise a gene or transgene that when expressed in sensory neurons, such as the DRG and/or trigeminal ganglia, results in silencing of activity of said sensory neurons. The silencing of activity of said sensory neurons results in the inhibition of chronic pain pathways. In an embodiment of the invention, the payload sequence comprises a potassium channel, such as KCNS1. In an embodiment of the invention, the payload sequence comprises a gene involved in the expression or delivery of opioid peptide genes. Examples of opioid peptide genes include proenkephalin (PENK) and prodynorphin. In an embodiment of the invention, dominant gene mutants that diminish sensory neuron activity may be comprised in the payload sequence. In an embodiment of the invention, dominant gene mutants that diminish sensory neuron activity, such as the ZFHX2 mutant p.R1913K, may be comprised in the payload sequence.

### Double-Stranded RNAs

Using known techniques and based on a knowledge of the sequence of the gene to be silenced, double-stranded RNA (dsRNA) molecules can be designed to silence the gene by sequence homology-based targeting of the gene's RNA. Such dsRNAs will typically be small interfering RNAs (siRNAs), usually in a stem-loop (“hairpin”) configuration, or micro-RNAs (miRNAs). The sequence of such dsRNAs will comprise a portion that corresponds with that of a portion of the mRNA encoding the gene. This portion will usually be 100% complementary to the target portion within the gene's mRNA but lower levels of complementarity (e.g. 90% or more or 95% or more) may also be used.

siRNAs

In one embodiment, the silencing mechanism comprises a small interfering RNA (siRNA). An siRNA acts by activating the RNAi-induced suppression complex. The siRNA molecules can be unmodified or modified and are capable of supressing gene expression. They are typically about 15 to 60 nucleotides in length. In some embodiments, the modified siRNA contains at least one 2′O-Me purine or pyrimidine nucleotide such as a 2′O-Me-guanosine, 2′O-Me-uridine, 2′O-Me-adenosine, and/or 2′O-Me-cytosine nucleotide. The modified nucleotides can be present in one strand (i.e., sense or antisense) or both strands of the siRNA. The siRNA sequences may have overhangs or blunt ends.

The modified siRNA may comprise from about 1% to about 100% (e.g., about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%) modified nucleotides in the double-stranded region of the siRNA duplex. In certain embodiments, one, two, three, four, five, six, seven, eight, nine, ten, or more of the nucleotides in the double-stranded region of the siRNA comprise modified nucleotides.

Suitable siRNA sequences can be identified using any means known in the art. Typically, the methods described in Elbashir et al., Nature, 411:494-498 (2001) and Elbashir et al., EMBO 1, 20:6877-6888 (2001) are combined with rational design rules set forth in Reynolds et al., Nature Biotech., 22(3):326-330 (2004).

Preferably, siRNA are chemically synthesized. The oligonucleotides that comprise the siRNA molecules of the invention can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997). The synthesis of oligonucleotides makes use of common nucleic acid protecting and coupling groups, such as dimethoxytrityl at the 5′-end and phosphoramidites at the 3′-end. Alternatively, siRNA molecules can be assembled from two distinct oligonucleotides, wherein one oligonucleotide comprises the sense strand and the other comprises the antisense strand of the siRNA. For example, each strand can be synthesized separately and joined together by hybridization or ligation following synthesis and/or deprotection. In certain other instances, siRNA molecules can be synthesized as a single continuous oligonucleotide fragment, where the self-complementary sense and antisense regions hybridize to form an siRNA duplex having hairpin secondary structure.

### CRISPR and Guide RNAs

In one embodiment, the silencing mechanism encompasses a mechanism of gene silencing by CRISPR (Clustered Regularly Interspaced Short Palindromic Repeats). In one embodiment, the mechanism of gene silencing by CRISPR involves the use of a guide RNA. The guide RNA may comprise a guide RNA sequence and a tracr RNA. The guide RNA sequence is capable of hybridizing to a target sequence in the DNA to be silenced. The tracr RNA is coupled to the guide RNA sequence. The guide RNA hybridises to the site of the allele and targets a CRISPR-Cas enzyme to said site.

In some embodiments, the guide RNA is between 10-30, or between 15-25, or between 15-20 nucleotides in length. In some embodiments one guide RNA is used. In some embodiments two guide RNAs are used. In some embodiments more than two guide RNAs are used.

Preferably the CRISPR-Cas enzyme is a Type II CRISPR enzyme, for example Cas-9 (CRISPR associated protein 9). In some preferred embodiments, the Cas-9 enzyme is SaCas-9.

The enzyme complexes with the guide RNA. In one embodiment, the complex targeted to the DNA sequence will bind by hybridization. In one embodiment, the enzyme is active and acts as an endonuclease to cleave the DNA either via activation of the non-homologous end-joining or homologous DNA repair pathway, resulting in a blunt end cut or a nick. In one embodiment, the use of guide RNA or RNAs and the CRISPR enzyme results in the deletion of essential elements of the gene to be silenced, resulting in a non-functional gene. In some embodiments, the gene is not transcribed. In some embodiments, the gene is not translated.

In a preferred embodiment of the invention, the gene to be silenced is the Nav1.7 gene SCN9A. In a preferred embodiment of the invention, the SCN9A gene is silenced by use of one or more of the guide RNAs of SEQ ID NO: 10, 11, and 12. In one preferred embodiment, the guide RNAs used are SEQ ID NOs: 10 and 11, and the enzyme is SaCas-9. In a preferred embodiment of the invention, the payload sequence comprises the SaCas-9 sequence as set out in SEQ ID NO: 13 and 16. In a preferred embodiment of the invention, the SCN9A gene is silenced by use of one or more of human guide RNAs.

In a preferred embodiment of the invention, the gene to be silenced is the Cav2.2 gene. In a preferred embodiment of the invention, the gene to be silenced is the Cav2.2 gene CACNAIB. In a preferred embodiment of the invention, the CACNAIB gene is silenced by use of one or more of human guide RNAs.

In another embodiment, the enzyme is targeted to the DNA of the gene to be silenced but the enzyme comprises one or more mutations that reduce or eliminate its endonuclease activity such that it does not edit the mutant allele but does prevent or reduce its transcription. An example of such an enzyme for use in the invention is dCas-9, which is catalytically dead. In a preferred embodiment, the dCas-9 is dSa-Cas9. In a preferred embodiment, the dCas-9 is dSa-Cas9 as set out in SEQ ID NO: 14. In a preferred embodiment of the invention, the payload sequence comprises the dSaCas-9 sequence of SEQ ID NO: 14.

In one embodiment dCas-9 is associated with a transcriptional repressor peptide that can knock down gene expression by interfering with transcription. In a preferred embodiment, the transcriptional repressor protein is Kruppel-associated box (KRAB). In a preferred embodiment, the transcriptional repressor protein is Kruppel-associated box (KRAB) as set out in SEQ ID NO: 15.

In a related embodiment, the enzyme can be engineered such that it is fused to a transcriptional repressor to reduce or disable its endonuclease function. The enzyme will be able to bind the guide RNA and be targeted to the DNA sequence, but no cleavage of the DNA takes place. The mutant allele may be suppressed, for example, by the shutting down of the promoter or blockage of RNA polymerase.

In a preferred embodiment the enzyme is dSaCas9-KRAB. In a preferred embodiment the enzyme is dSaCas9-KRAB and comprises the sequences of SEQ ID NOs: 14 and 15. In another preferred embodiment, the guide RNA used is SEQ ID NO: 12 and the enzyme is dSaCas9-KRAB. In a preferred embodiment of the invention, the payload sequence comprises a dSaCas9-KRAB sequence. In a preferred embodiment of the invention, the payload sequence comprises the dSaCas9 and KRAB sequences of SEQ ID NOs: 14 and 15. In a preferred embodiment of the invention, the payload sequence comprises the dSaCas9-KRAB sequence of SEQ ID NO: 17.

In another embodiment, the transcription repressor may be bound to the tracr sequence. Functional domains can be attached to the tracr sequence by incorporating protein-binding RNA aptamer sequences, as described in Konermann et al (Genome-scale transcriptional activation by an engineered CRISPR-Cas9 complex, Nature, Vol 517(7536):583-8, 2014). The transcription repressor-tracr sequence complex may be used to target other moieties to a precise gene location as desired.

In another embodiment, the CRISPR mechanism of silencing involves CRISPR base editors to knock out genes by changing single nucleotides to create stop codons (CRISPR-STOP method (Kuscu et al. 2017)).

In another embodiment, the CRISPR mechanism of silencing involves CRISPR activation mediated upregulation of a gene, said upregulation resulting in the silencing of a target gene as described herein. Thus, in an embodiment of the invention, the payload sequence can comprise a dSaCas9-VPR sequence.

ZFPs

In another embodiment of the invention, the mechanism of silencing encompasses the use of zinc finger proteins (ZFPs, otherwise known as zinc finger nucleases or ZFNs). A ZFP is a heterodimer in which each subunit contains a zinc finger domain and a FokI endonuclease domain. ZFPs constitute the largest individual family of transcriptional modulators known for higher organisms. In certain embodiments, the payload sequence comprises a DNA-binding domain made up of Cys2His2 zinc fingers fused to a KRAB repressor. In a preferred embodiment of the invention, the payload sequence comprises a zinc-finger-KRAB sequence.

### Talens

In another embodiment of the invention, the mechanism of silencing encompasses the use of transcription activator-like effector nucleases (TALENs). TALENs comprise a non-specific DNA-cleaving nuclease fused to a DNA-binding domain that can be customised so that TALENs can target a sequence of interest to be silenced (Joung and Sander, 2013). In certain embodiments, the payload sequence comprises a TALEN sequence.

### Dreadds

In another embodiment of the invention, the mechanism of silencing encompasses the use of designer receptor exclusively activated by designer drugs (DREADDs). DREADDs are families of designer G-protein-coupled receptors (GPCRs) built specifically to allow for precise spatiotemporal control of GPCR signalling in vivo that regulate neuronal excitability. The DREADD system has been used to selectively inhibit or activate neuronal electrical activity (Magnus et al., 2019). In one embodiment of the invention, the payload sequence comprises a DREADD.

### Promoters of the Invention

The invention provides a heterologous, exogenous Advillin (Avil) promoter sequence, as described in more detail below. The Advillin promoter sequence of the present invention drives expression of payload sequences in sensory neurons. The Advillin promoter sequence of the present invention drives expression of payload sequences in sensory neurons such as the DRG. The Advillin promoter sequence of the present invention drives expression of payload sequences in sensory neurons such as the trigeminal ganglia. In a preferred embodiment, the invention provides an Advillin promoter fragment of no more than 500 nucleotides in length that comprises SEQ ID NO: 1.

### Expression Constructs of the Invention

An expression construct may be defined as a polynucleotide sequence capable of driving protein expression from a polynucleotide sequence containing a coding sequence. The expression constructs of the present invention comprises the Advillin promoter of the invention.

The expression construct of the invention may comprise in a 5′ to 3′ direction:

(a) an Advillin (Avil) promoter consisting of a sequence of no more than 500 contiguous nucleotides comprising SEQ ID NO:1; and
(b) a payload sequence,
wherein the promoter of (a) is operably linked to the payload sequence of (b).

An expression construct of the present invention may also include additional nucleotide sequences naturally found in an Avil promoter region. The expression construct of the invention may comprise an Advillin (Avil) promoter consisting of a sequence of no more than 500 contiguous nucleotides comprising SEQ ID NO:1, wherein the 500 contiguous nucleotides contain sequences from SEQ ID NO: 6, 7, 8 or 9.

The expression construct of the invention may comprise an Advillin (Avil) promoter consisting of a sequence of no more than 500, 490, 480,470, 460, 450, 440, 430, 420, 410 or 400 contiguous nucleotides comprising SEQ ID NO:1.

The expression construct of the invention may comprise an Advillin (Avil) promoter consisting of a sequence of no more than 500 contiguous nucleotides from SEQ ID NO: 6 comprising SEQ ID NO: 2. The expression construct of the invention may comprise an Advillin (Avil) promoter consisting of a sequence of no more than 480 contiguous nucleotides from SEQ ID NO: 6 comprising SEQ ID NO: 2. The expression construct of the invention may comprise an Advillin (Avil) promoter consisting of a sequence of no more than 450 contiguous nucleotides from SEQ ID NO: 6 comprising SEQ ID NO: 2. The expression construct of the invention may comprise an Advillin (Avil) promoter consisting of a sequence of no more than 430 contiguous nucleotides from SEQ ID NO: 6 comprising SEQ ID NO: 2. The expression construct of the invention may comprise an Advillin (Avil) promoter consisting of a sequence of no more than 410 contiguous nucleotides from SEQ ID NO: 6 comprising SEQ ID NO: 2. The expression construct of the invention may comprise an Advillin (Avil) promoter consisting of a sequence of no more than 400 contiguous nucleotides from SEQ ID NO: 6 comprising SEQ ID NO: 2. The expression construct of the invention may comprise an Advillin (Avil) promoter consisting of SEQ ID NO: 2.

The expression construct of the invention may comprise an Advillin (Avil) promoter consisting of a sequence of no more than 500 contiguous nucleotides from SEQ ID NO: 7 comprising SEQ ID NO: 3. The expression construct of the invention may comprise an Advillin (Avil) promoter consisting of a sequence of no more than 480 contiguous nucleotides from SEQ ID NO: 7 comprising SEQ ID NO: 3. The expression construct of the invention may comprise an Advillin (Avil) promoter consisting of a sequence of no more than 450 contiguous nucleotides from SEQ ID NO: 7 comprising SEQ ID NO: 3. The expression construct of the invention may comprise an Advillin (Avil) promoter consisting of a sequence of no more than 430 contiguous nucleotides from SEQ ID NO: 7 comprising SEQ ID NO: 3. The expression construct of the invention may comprise an Advillin (Avil) promoter consisting of a sequence of no more than 410 contiguous nucleotides from SEQ ID NO: 7 comprising SEQ ID NO: 3. The expression construct of the invention may comprise an Advillin (Avil) promoter consisting of a sequence of no more than 400 contiguous nucleotides from SEQ ID NO: 7 comprising SEQ ID NO: 3. The expression construct of the invention may comprise an Advillin (Avil) promoter consisting of SEQ ID NO: 3.

The expression construct of the invention may comprise an Advillin (Avil) promoter consisting of a sequence of no more than 500 contiguous nucleotides from SEQ ID NO: 8 comprising SEQ ID NO: 4. The expression construct of the invention may comprise an Advillin (Avil) promoter consisting of a sequence of no more than 480 contiguous nucleotides from SEQ ID NO: 8 comprising SEQ ID NO: 4. The expression construct of the invention may comprise an Advillin (Avil) promoter consisting of a sequence of no more than 450 contiguous nucleotides from SEQ ID NO: 8 comprising SEQ ID NO: 4. The expression construct of the invention may comprise an Advillin (Avil) promoter consisting of a sequence of no more than 430 contiguous nucleotides from SEQ ID NO: 8 comprising SEQ ID NO: 4. The expression construct of the invention may comprise an Advillin (Avil) promoter consisting of a sequence of no more than 410 contiguous nucleotides from SEQ ID NO: 8 comprising SEQ ID NO: 4. The expression construct of the invention may comprise an Advillin (Avil) promoter consisting of a sequence of no more than 400 contiguous nucleotides from SEQ ID NO: 8 comprising SEQ ID NO: 4. The expression construct of the invention may comprise an Advillin (Avil) promoter consisting of SEQ ID NO: 4.

The expression construct of the invention may comprise an Advillin (Avil) promoter consisting of a sequence of no more than 500 contiguous nucleotides from SEQ ID NO: 9 comprising SEQ ID NO: 5. The expression construct of the invention may comprise an Advillin (Avil) promoter consisting of a sequence of no more than 480 contiguous nucleotides from SEQ ID NO: 9 comprising SEQ ID NO: 5. The expression construct of the invention may comprise an Advillin (Avil) promoter consisting of a sequence of no more than 450 contiguous nucleotides from SEQ ID NO: 9 comprising SEQ ID NO: 5. The expression construct of the invention may comprise an Advillin (Avil) promoter consisting of a sequence of no more than 430 contiguous nucleotides from SEQ ID NO: 9 comprising SEQ ID NO: 5. The expression construct of the invention may comprise an Advillin (Avil) promoter consisting of a sequence of no more than 410 contiguous nucleotides from SEQ ID NO: 9 comprising SEQ ID NO: 5. The expression construct of the invention may comprise an Advillin (Avil) promoter consisting of a sequence of no more than 400 contiguous nucleotides from SEQ ID NO: 9 comprising SEQ ID NO: 5. The expression construct of the invention may comprise an Advillin (Avil) promoter consisting of SEQ ID NO: 5.

An expression construct of the present invention may also include additional nucleotide sequences not naturally found in the Avil promoter region. An expression construct of the present invention may also include additional nucleotide sequences 5′ to the promoter sequence of Avil, 3′ to the promoter sequence of Avil but 5′ to payload, and/or 3′ to payload.

The expression constructs of the present invention can also be used in tandem with other regulatory elements such as one or more further promoters or enhancers or locus control regions (LCRs).

Further expression constructs of the invention may comprise promoters that differ in sequence from the Avil promoter sequences above but retain the ability to express payload sequences in sensory neurons such as the DRG and/or the trigeminal ganglia. Such sequences have at least 75%, at least 80%, at least 85%, at least 90%, at least 95%, at least 98% or at least 99% sequence identity to a sequence of contiguous nucleotides from SEQ ID NOs: 1, 2, 3, 4, or 5, or have at least 75%, at least 80%, at least 85%, at least 90%, at least 95%, at least 98% or at least 99% sequence identity to SEQ ID NOs: 1, 2, 3, 4, or 5. In an embodiment of the invention such sequences are no more than 500, 490, 480, 470, 460, 450, 440, 430, 420, 410 or 400 nucleotides in length and have at least 75%, at least 80%, at least 85%, at least 90%, at least 95%, at least 98% or at least 99% sequence identity to SEQ ID NOs: 1, 2, 3, 4, or 5.

Retaining the ability to express the payload sequence in sensory neurons such as the DRG and/or the trigeminal ganglia can be measured by any suitable standard technique known to the person skilled in the art, for example, RNA expression levels can be measured by quantitative real-time PCR.

Thus the expression construct of the invention may also comprise in a 5′ to 3′ direction:

(a) a sequence of no more than 500 contiguous nucleotides having 90% sequence identity to SEQ ID NO: 1, that retains the ability to express a payload sequence in sensory neurons such as dorsal root ganglion (DRG) neurons and/or the trigeminal ganglia;
(b) a payload sequence,
wherein the promoter of (a) is operably linked to the payload sequence of (b).

In an embodiment of the invention the expression construct of the invention may also comprise in a 5′ to 3′ direction:

(a) a sequence of no more than 500 contiguous nucleotides having 90% sequence identity to SEQ ID NO: 1, that retains the ability to express a payload sequence in dorsal root ganglion (DRG) neurons and/or the trigeminal ganglia;
(b) a payload sequence,
wherein the promoter of (a) is operably linked to the payload sequence of (b).

Sequence identity may be calculated using any suitable algorithm. For example the PILEUP and BLAST algorithms can be used to calculate identity or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10. Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001. Alternatively, the UWGCG Package provides the BESTFIT program which can be used to calculate identity (for example used on its default settings) (Devereux et al (1984) Nucleic Acids Research 12, 387-395).

Vectors

The present invention provides vectors comprising the expression constructs of the present invention. The vector may be of any type, for example it may be a plasmid vector or a minicircle DNA.

The present invention encompasses the delivery of the vector of the invention to a cell. In an embodiment of the invention the cell is a sensory neuron cell, such as a DRG cell or a trigeminal ganglia cell. In an embodiment of the invention, the vector may be delivered to the cell via a non-viral delivery method. In an embodiment of the invention, the vector may be delivered to the cell in vesicles. In an embodiment of the invention, the vector may be delivered to the cell in nanoparticles. In an embodiment of the invention, the vector may be delivered to the cell in exosomes. In an embodiment of the invention the vector is packaged in a vesicle, liposome, exosome or in a nanoparticle.

Typically, vectors of the invention are however viral vectors. The viral vector may be based on the herpes simplex virus, adenovirus or lentivirus. The viral vector may be an adeno-associated virus (AAV) vector or a derivative thereof.

The viral vector derivative may be a chimeric, shuffled or capsid modified derivative.

The viral vector may comprise an AAV genome from a naturally derived serotype, isolate or Glade of AAV.

The serotype may for example be AAV2, AAV5 or AAV8.

The efficacy of gene therapy is, in general, dependent upon adequate and efficient delivery of the donated DNA. This process is usually mediated by viral vectors. Adeno-associated viruses (AAV), a member of the parvovirus family, are commonly used in gene therapy. Wild-type AAV, containing viral genes, insert their genomic material into chromosome 19 of the host cell (Kotin, et al. 1990). The AAV single-stranded DNA genome comprises two inverted terminal repeats (ITRs) and two open reading frames, containing structural (cap) and packaging (rep) genes (Hermonat et al. 1984).

For therapeutic purposes, the only sequences required in cis, in addition to the therapeutic gene, are the ITRs. The AAV virus is therefore modified: the viral genes are removed from the genome, producing recombinant AAV (rAAV). This contains only the therapeutic gene, the two ITRs. The removal of the viral genes renders rAAV incapable of actively inserting its genome into the host cell DNA. Instead, the rAAV genomes fuse via the ITRs, forming circular, episomal structures, or insert into pre-existing chromosomal breaks. For viral production, the structural and packaging genes, now removed from the rAAV, are supplied in trans, in the form of a helper plasmid.

AAV is a particularly attractive vector as it is generally non-pathogenic; the majority of people have been infected with this virus during their life with no adverse effects (Erles et al. 1999). Despite this, there are several drawbacks to the use of rAAV in gene therapy, although the majority of these only apply to systemic administration of rAAV. Nevertheless, it is important to acknowledge these potential limitations. Infection can trigger the following immunological responses:

As the majority of the human population is seropositive for AAV, neutralising antibodies against rAAV can impair gene delivery (Moskalenko et al. 2000; Sun et al. 2003). Systemically delivered rAAV can trigger a capsid protein-directed T-cell response, leading to the apoptosis of transduced cells (Manno et al. 2006).

rAAV vectors can trigger complement activation (Zaiss et al. 2008).

As the rAAV delivery is generally unspecific, the vector can accumulate in the liver (Michelfelder et al. 2009).

AAV vectors are limited by a relatively small packaging capacity of roughly 4.8 kb and a slow onset of expression following transduction (Dong et al. 1996).

Most vector constructs are based on the AAV serotype 2 (AAV2). AAV2 binds to the target cells via the heparin sulphate proteoglycan receptor (Summerford and and Samulski 1998). The AAV2 genome, like those of all AAV serotypes, can be enclosed in a number of different capsid proteins. AAV2 can be packaged in its natural AAV2 capsid (AAV2/2) or it can be pseudotyped with other capsids (e.g. AAV2 genome in AAV1 capsid; AAV2/1, AAV2 genome in AAV5 capsid; AAV2/5 and AAV2 genome in AAV8 capsid; AAV2/8).

rAAV transduces cells via serotype specific receptor-mediated endocytosis. A major factor influencing the kinetics of rAAV transgene expression is the rate of virus particle uncoating within the endosome (Thomas et al. 2004). This, in turn, depends upon the type of capsid enclosing the genetic material (Ibid.). After uncoating the linear single-stranded rAAV genome is stabilised by forming a double-stranded molecule via de novo synthesis of a complementary strand (Vincent-Lacaze et al. 1999). The use of self-complementary DNA may bypass this stage by producing double-stranded transgene DNA. Natkunarajah et al. found that self-complementary AAV2/8 gene expression was of faster onset and higher amplitude, compared to single-stranded AAV2/8 (2008). Thus, by circumventing the time lag associated with second-strand synthesis, gene expression levels are increased, when compared to transgene expression from standard single-stranded constructs. Subsequent studies investigating the effect of self-complementary DNA in other AAV pseudotypes (e.g. AAV2/5) have produced similar results (Kong et al. 2010; Petersen-Jones et al. 2009). One caveat to this technique is that, as AAV has a packaging capacity of approximately 4.8 kb, the self-complementary recombinant genome must be appropriately sized (i.e. 2.3 kb or less).

In addition to modifying packaging capacity, pseudotyping the AAV2 genome with other AAV capsids can alter cell specificity and the kinetics of transgene expression.

### Aa V Genome

The vector of the present invention may comprise an adeno-associated virus (AAV) genome or a derivative thereof.

An AAV genome is a polynucleotide sequence which encodes functions needed for production of an AAV viral particle. These functions include those operating in the replication and packaging cycle for AAV in a host cell, including encapsidation of the AAV genome into an AAV viral particle. Naturally occurring AAV viruses are replication-deficient and rely on the provision of helper functions in trans for completion of a replication and packaging cycle. Accordingly and with the additional removal of the AAV rep and cap genes, the AAV genome of the vector of the invention is replication-deficient.

The AAV genome may be in single-stranded form, either positive or negative-sense, or alternatively in double-stranded form. The use of a double-stranded form allows bypass of the DNA replication step in the target cell and so can accelerate transgene expression.

The AAV genome may be from any naturally derived serotype or isolate or Glade of AAV. As is known to the skilled person, AAV viruses occurring in nature may be classified according to various biological systems.

Commonly, AAV viruses are referred to in terms of their serotype. A serotype corresponds to a variant subspecies of AAV which owing to its profile of expression of capsid surface antigens has a distinctive reactivity which can be used to distinguish it from other variant subspecies. Typically, a virus having a particular AAV serotype does not efficiently cross-react with neutralising antibodies specific for any other AAV serotype. AAV serotypes include AAV1, AAV2, AAV3, AAV4, AAV5, AAV6, AAV7, AAV5, AAV5, AAV10 and AAV11, also recombinant serotypes, such as Rec2 and Rec3, recently identified from primate brain. In vectors of the invention, the genome may be derived from any AAV serotype. The capsid may also be derived from any AAV serotype. The genome and the capsid may be derived from the same serotype or different serotypes.

In vectors of the invention, it is preferred that the genome is derived from AAV serotype 2 (AAV2), AAV serotype 4 (AAV4), AAV serotype 5 (AAV5) or AAV serotype 8 (AAV8). It is most preferred that the genome is derived from AAV2 but other serotypes of particular interest for use in the invention include AAV4, AAV5 and AAV8. It is preferred that the capsid is derived from AAV1 or AAV9.

In a preferred embodiment of the invention the genome is derived from AAV serotype 2 (AAV2) and the capsid is derived from AAV1 or AAV9, i.e. AAV2/1 or AAV2/9.

Reviews of AAV serotypes may be found in Choi et al (Curr Gene Ther. 2005; 5(3); 299-310) and Wu et al (Molecular Therapy. 2006; 14(3), 316-327). The sequences of AAV genomes or of elements of AAV genomes including ITR sequences, rep or cap genes for use in the invention may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV viruses may also be referred to in terms of clades or clones. This refers to the phylogenetic relationship of naturally derived AAV viruses, and typically to a phylogenetic group of AAV viruses which can be traced back to a common ancestor, and includes all descendants thereof. Additionally, AAV viruses may be referred to in terms of a specific isolate, i.e. a genetic isolate of a specific AAV virus found in nature. The term genetic isolate describes a population of AAV viruses which has undergone limited genetic mixing with other naturally occurring AAV viruses, thereby defining a recognisably distinct population at a genetic level.

Examples of clades and isolates of AAV that may be used in the invention include:

Clade A: AAV1 NC_002077, AF063497, AAV6 NC_001862, Hu. 48 AY530611, Hu 43 AY530606, Hu 44 AY530607, Hu 46 AY530609

Clade B: Hu. 19 AY530584, Hu. 20 AY530586, Hu 23 AY530589, Hu22 AY530588, Hu24 AY530590, Hu21 AY530587, Hu27 AY530592, Hu28 AY530593, Hu 29 AY530594, Hu63 AY530624, Hu64 AY530625, Hu13 AY530578, Hu56 AY530618, Hu57 AY530619, Hu49 AY530612, Hu58 AY530620, Hu34 AY530598, Hu35 AY530599, AAV2 NC_001401, Hu45 AY530608, Hu47 AY530610, Hu51 AY530613, Hu52 AY530614, Hu T41 AY695378, Hu S17 AY695376, Hu T88 AY695375, Hu T71 AY695374, Hu T70 AY695373, Hu T40 AY695372, Hu T32 AY695371, Hu T17 AY695370, Hu LG15 AY695377,

Clade C: Hu9 AY530629, Hu10 AY530576, Hu11 AY530577, Hu53 AY530615, Hu55 AY530617, Hu54 AY530616, Hu7 AY530628, Hu18 AY530583, Hu15 AY530580, Hu16 AY530581, Hu25 AY530591, Hu60 AY530622, Ch5 AY243021, Hu3 AY530595, Hu1 AY530575, Hu4 AY530602 Hu2, AY530585, Hu61 AY530623

Clade D: Rh62 AY530573, Rh48 AY530561, Rh54 AY530567, Rh55 AY530568, Cy2 AY243020, AAV7 AF513851, Rh35 AY243000, Rh37 AY242998, Rh36 AY242999, Cy6 AY243016, Cy4 AY243018, Cy3 AY243019, Cy5 AY243017, Rh13 AY243013

Clade E: Rh38 AY530558, Hu66 AY530626, Hu42 AY530605, Hu67 AY530627, Hu40 AY530603, Hu41 AY530604, Hu37 AY530600, Rh40 AY530559, Rh2 AY243007, Bb1 AY243023, Bb2 AY243022, Rh10 AY243015, Hu17 AY530582, Hu6 AY530621, Rh25 AY530557, Pi2 AY530554, Pi1 AY530553, Pi3 AY530555, Rh57 AY530569, Rh50 AY530563, Rh49 AY530562, Hu39 AY530601, Rh58 AY530570, Rh61 AY530572, Rh52 AY530565, Rh53 AY530566, Rh51 AY530564, Rh64 AY530574, Rh43 AY530560, AAV8 AF513852, Rh8 AY242997, Rh1 AY530556

Clade F: Hu14 (AAV9) AY530579, Hu31 AY530596, Hu32 AY530597, Clonal Isolate AAV5 Y18065, AF085716, AAV 3 NC_001729, AAV 3B NC_001863, AAV4 NC_001829, Rh34 AY243001, Rh33 AY243002, Rh32 AY243003/

The skilled person can select an appropriate serotype, Glade, clone or isolate of AAV for use in the present invention on the basis of their common general knowledge.

It should be understood however that the invention also encompasses use of an AAV genome of other serotypes that may not yet have been identified or characterised. The AAV serotype determines the tissue specificity of infection (or tropism) of an AAV virus.

Typically, the AAV genome of a naturally derived serotype or isolate or Glade of AAV comprises at least one inverted terminal repeat sequence (ITR). Vectors of the invention typically comprise two ITRs, preferably one at each end of the genome. An ITR sequence acts in cis to provide a functional origin of replication, and allows for integration and excision of the vector from the genome of a cell. Preferred ITR sequences are those of AAV2 and variants thereof. The AAV genome typically comprises packaging genes, such as rep and/or cap genes which encode packaging functions for an AAV viral particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV viral particle. Capsid variants are discussed below.

Preferably the AAV genome will be derivatised for the purpose of administration to patients. Such derivatisation is standard in the art and the present invention encompasses the use of any known derivative of an AAV genome, and derivatives which could be generated by applying techniques known in the art. Derivatisation of the AAV genome and of the AAV capsid are reviewed in Coura and Nardi (Virology Journal, 2007, 4:99), and in Choi et al and Wu et al, referenced above.

Derivatives of an AAV genome include any truncated or modified forms of an AAV genome which allow for expression of a Rep-1 transgene from a vector of the invention in vivo. Typically, it is possible to truncate the AAV genome significantly to include minimal viral sequence yet retain the above function. This is preferred for safety reasons to reduce the risk of recombination of the vector with wild-type virus, and also to avoid triggering a cellular immune response by the presence of viral gene proteins in the target cell.

Typically, a derivative will include at least one inverted terminal repeat sequence (ITR), preferably more than one ITR, such as two ITRs or more. One or more of the ITRs may be derived from AAV genomes having different serotypes, or may be a chimeric or mutant ITR. A preferred mutant ITR is one having a deletion of a trs (terminal resolution site). This deletion allows for continued replication of the genome to generate a single-stranded genome which contains both coding and complementary sequences i.e. a self-complementary AAV genome. This allows for bypass of DNA replication in the target cell, and so enables accelerated transgene expression.

The one or more ITRs will preferably flank the expression construct cassette containing the promoter and transgene of the invention. The inclusion of one or more ITRs is preferred to aid packaging of the vector of the invention into viral particles. In preferred embodiments, ITR elements will be the only sequences retained from the native AAV genome in the derivative. Thus, a derivative will preferably not include the rep and/or cap genes of the native genome and any other sequences of the native genome. This is preferred for the reasons described above, and also to reduce the possibility of integration of the vector into the host cell genome. Additionally, reducing the size of the AAV genome allows for increased flexibility in incorporating other sequence elements (such as regulatory elements) within the vector in addition to the transgene.

With reference to the AAV2 genome, the following portions could therefore be removed in a derivative of the invention: One inverted terminal repeat (ITR) sequence, the replication (rep) and capsid (cap) genes. However, in some embodiments, including in vitro embodiments, derivatives may additionally include one or more rep and/or cap genes or other viral sequences of an AAV genome.

A derivative may be a chimeric, shuffled or capsid-modified derivative of one or more naturally occurring AAV viruses. The invention encompasses the provision of capsid protein sequences from different serotypes, clades, clones, or isolates of AAV within the same vector. The invention encompasses the packaging of the genome of one serotype into the capsid of another serotype i.e. pseudotyping.

Chimeric, shuffled or capsid-modified derivatives will be typically selected to provide one or more desired functionalities for the viral vector. Thus, these derivatives may display increased efficiency of gene delivery, decreased immunogenicity (humoral or cellular), an altered tropism range and/or improved targeting of a particular cell type compared to an AAV viral vector comprising a naturally occurring AAV genome, such as that of AAV2. Increased efficiency of gene delivery may be effected by improved receptor or co-receptor binding at the cell surface, improved internalisation, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are cotransfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence.

The unrelated protein or peptide may advantageously be one which acts as a ligand for a particular cell type, thereby conferring improved binding to a target cell or improving the specificity of targeting of the vector to a particular cell population.

The unrelated protein may also be one which assists purification of the viral particle as part of the production process i.e. an epitope or affinity tag. The site of insertion will typically be selected so as not to interfere with other functions of the viral particle e.g. internalisation, trafficking of the viral particle. The skilled person can identify suitable sites for insertion based on their common general knowledge. Particular sites are disclosed in Choi et al, referenced above.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

The vector of the invention takes the form of a viral vector comprising the expression constructs of the invention.

For the avoidance of doubt, the invention also provides an AAV viral particle comprising a vector of the invention. The AAV particles of the invention include transcapsidated forms wherein an AAV genome or derivative having an ITR of one serotype is packaged in the capsid of a different serotype. The AAV particles of the invention also include mosaic forms wherein a mixture of unmodified capsid proteins from two or more different serotypes makes up the viral envelope. The AAV particle also includes chemically modified forms bearing ligands adsorbed to the capsid surface. For example, such ligands may include antibodies for targeting a particular cell surface receptor.

The present invention encompasses the delivery of the viral vector of the invention to a cell. In an embodiment of the invention the cell is a sensory neuron cell, such as a DRG cell or a trigeminal ganglia cell. In an embodiment of the invention, the viral vector may be delivered to the cell in vesicles. In an embodiment of the invention, the viral vector may be delivered to the cell in nanoparticles. In an embodiment of the invention, the viral vector may be delivered to the cell in exosomes.

The invention additionally provides a host cell comprising a vector or AAV viral particle of the invention.

Preparation of Vector

The vector of the invention may be prepared by standard means known in the art for provision of vectors for gene therapy. Thus, well established public domain transfection, packaging and purification methods can be used to prepare a suitable vector preparation.

As discussed above, a vector of the invention may comprise the full genome of a naturally occurring AAV virus in addition to a promoter of the invention or a variant thereof. However, commonly a derivatised genome will be used, for instance a derivative which has at least one inverted terminal repeat sequence (ITR), but which may lack any AAV genes such as rep or cap.

In such embodiments, in order to provide for assembly of the derivatised genome into an AAV viral particle, additional genetic constructs providing AAV and/or helper virus functions will be provided in a host cell in combination with the derivatised genome. These additional constructs will typically contain genes encoding structural AAV capsid proteins i.e. cap, VP1, VP2, VP3, and genes encoding other functions required for the AAV life cycle, such as rep. The selection of structural capsid proteins provided on the additional construct will determine the serotype of the packaged viral vector.

A particularly preferred packaged viral vector for use in the invention comprises a derivatised genome of AAV2 in combination with AAV 1 or AAV9 capsid proteins.

As mentioned above, AAV viruses are replication incompetent and so helper virus functions, preferably adenovirus helper functions will typically also be provided on one or more additional constructs to allow for AAV replication.

All of the above additional constructs may be provided as plasmids or other episomal elements in the host cell, or alternatively one or more constructs may be integrated into the genome of the host cell.

Expression constructs and vectors of the invention have the ability to treat, prevent, ameliorate, slow the progression or abrogate chronic pain.

The properties of the expression constructs and vectors of the invention can also be tested using techniques known by the person skilled in the art. In particular, a sequence of the invention can be assembled into a vector of the invention and delivered to a test animal, such as a mouse, and the effects observed and compared to a control.

### Methods of Therapy and Medical Uses

The expression constructs and vectors of the invention may be used in the treatment or prevention of chronic pain. The expression constructs and vectors of the invention may be used in the amelioration of chronic pain. The expression constructs and vectors of the invention may be used in the treatment or prevention, or amelioration, of conditions involving chronic pain. This provides a means whereby the conditions can be treated, arrested, palliated or prevented.

The conditions to be treated by the invention include chronic pain such as cancer pain, cancer-associated bone pain, rheumatoid arthritis and osteoarthritis, trigeminal neuralgia, headache, migraine, fibromyalgia, diabetic neuropathy and other neuropathy associated pain, neuropathic pain and idiopathic pain. In preferred embodiments the conditions to be treated include rare diseases like erythromelalgia and paroxysmal extreme pain disorder.

The vectors of the invention can be used to treat chronic pain whilst not affecting the functionally of acute pain pathways. Not affecting the functionality can be defined as a patient being able to detect acute pain to an extent that allows for remedial action to be taken.

The invention therefore provides a pharmaceutical composition comprising the vector of the invention and a pharmaceutically acceptable carrier.

The invention also provides a vector for use in a method of treating or preventing chronic pain.

The invention also provides the use of a vector of the invention in the manufacture of a medicament for the treatment or prevention of chronic pain.

The invention also provides a method of treating or preventing chronic pain in a patient in need thereof comprising administering a therapeutically effective amount of a vector of the invention to the patient.

By using the Avil promoters in the expression constructs of the invention, expression of payload sequences can be achieved in sensory neurons such as the DRG and/or the trigeminal ganglia. Thus the expression constructs and vectors of the present invention can be used to silence target genes involved in chronic pain pathways to treat or prevent chronic pain.

In general, parenteral routes of delivery of vectors of the invention, such as intravenous (IV) or intracerebroventricular (ICV) administration, typically by injection, are preferred. In a preferred embodiment of the invention, the delivery route is intrathecal (IT).

The invention therefore also provides a method of treating or preventing chronic pain in a patient in need thereof, comprising administering a therapeutically effective amount of a vector of the invention to the patient by a parenteral route of administration. Accordingly, chronic pain is thereby treated or prevented in said patient.

In a related aspect, the invention provides for use of a vector of the invention in a method of treating or preventing chronic pain by administering said vector to a patient by a parenteral route of administration. Additionally, the invention provides the use of a vector of the invention in the manufacture of a medicament for the treatment or prevention of chronic pain by a parenteral route of administration.

In all these embodiments, the vector of the invention may be administered in order to prevent the onset of chronic pain.

In all these embodiments, the vector of the invention may be administered in order to prevent the onset of one or more symptoms of chronic pain.

A prophylactically effective amount of the vector is administered to such a subject. A prophylactically effective amount is an amount which prevents the onset of one or more symptoms of chronic pain.

Alternatively, the vector may be administered once the symptoms of chronic pain have appeared in a subject i.e. to cure existing symptoms of the chronic pain. A therapeutically effective amount of the antagonist is administered to such a subject. A therapeutically effective amount is an amount which is effective to ameliorate one or more symptoms of the chronic pain.

The subject may be male or female. The subject is preferably identified as being at risk of, or having, chronic pain.

The administration of the vector is typically by a parenteral route of administration. Parenteral routes of administration encompass intravenous (IV), intramuscular (IM), subcutaneous (SC), epidural (E), intracerebral (IC), intracerebroventricular (ICV) intrathecal (IT) and intradermal (ID) administration.

The dose of a vector of the invention may be determined according to various parameters, especially according to the age, weight and condition of the patient to be treated; the route of administration; and the required regimen. Again, a physician will be able to determine the required route of administration and dosage for any particular patient. For example, a suitable dose of a vector of the present invention may be in the range of 6.7×10¹³ vg/kg to 2.0 x10″ vg/kg, where vg=viral genome.

The dose may be provided as a single dose, but may be repeated in cases where vector may not have targeted the correct region. The treatment is preferably a single permanent injection, but repeat injections, for example in future years and/or with different AAV serotypes may be considered.

### Host Cells

Any suitable host cell can be used to produce the vectors of the invention. In general, such cells will be transfected mammalian cells but other cell types, e.g. insect cells, can also be used. In terms of mammalian cell production systems, HEK293 and HEK293T are preferred for AAV vectors. BHK or CHO cells may also be used.

### Pharmaceutical Compositions and Dosages

The vector of the invention can be formulated into pharmaceutical compositions. These compositions may comprise, in addition to the vector, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may be determined by the skilled person according to the route of administration.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution or glycols such as ethylene glycol, propylene glycol or polyethylene glycol may be included. In some cases, a surfactant, such as pluronic acid (PF68) 0.001% may be used.

For injection at the site of affliction, the active ingredient will be in the form of an aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection, Hartmann's solution. Preservatives, stabilisers, buffers, antioxidants and/or other additives may be included, as required.

For delayed release, the vector may be included in a pharmaceutical composition which is formulated for slow release, such as in microcapsules formed from biocompatible polymers or in liposomal carrier systems according to methods known in the art.

Dosages and dosage regimes can be determined within the normal skill of the medical practitioner responsible for administration of the composition.

### Combination Therapies

The expression constructs, vectors and/or pharmaceutical compositions can be used in combination with any other therapy for the treatment or prevention of chronic pain.

The expression constructs, vectors and/or pharmaceutical compositions can also be used in combination with any other therapy for the treatment or prevention of chronic pain.

### Kits

The expression constructs, vectors and/or pharmaceutical compositions can be packaged into a kit.

### Embodiments of the Invention

Additional embodiments of the invention include:

1. An expression construct comprising in a 5′ to 3′ direction:
(a) (i) an Advillin (Avil) promoter consisting of:
(1) a sequence of no more than 500 contiguous nucleotides from SEQ ID NO: 6 comprising SEQ ID NO: 2, or
(2) a sequence of no more than 500 contiguous nucleotides comprising a sequence having 90% sequence identity to SEQ ID NO: 6, that retains the ability to express a payload sequence in dorsal root ganglion (DRG) neurons or trigeminal ganglia; and
   (b) a payload sequence,
   wherein the promoter of (a) is operably linked to the payload sequence of (b).
   2. An expression construct comprising in a 5′ to 3′ direction:
   (a) (i) an Advillin (Avil) promoter consisting of:
(1) a sequence of no more than 500 contiguous nucleotides from SEQ ID NO: 7 comprising SEQ ID NO: 3, or
(2) a sequence of no more than 500 contiguous nucleotides comprising a sequence having 90% sequence identity to SEQ ID NO: 7, that retains the ability to express a payload sequence in dorsal root ganglion (DRG) neurons or trigeminal ganglia; and
   (b) a payload sequence,
   wherein the promoter of (a) is operably linked to the payload sequence of (b).
   3. An expression construct comprising in a 5′ to 3′ direction:
   (a) (i) an Advillin (Avil) promoter consisting of:
(1) a sequence of no more than 500 contiguous nucleotides from SEQ ID NO: 8 comprising SEQ ID NO: 4, or
(2) a sequence of no more than 500 contiguous nucleotides comprising a sequence having 90% sequence identity to SEQ ID NO: 8, that retains the ability to express a payload sequence in dorsal root ganglion (DRG) neurons or trigeminal ganglia; and
   (b) a payload sequence,
   wherein the promoter of (a) is operably linked to the payload sequence of (b).
   4. An expression construct comprising in a 5′ to 3′ direction:
   (a) (i) an Advillin (Avil) promoter consisting of:
(1) a sequence of no more than 500 contiguous nucleotides from SEQ ID NO: 9 comprising SEQ ID NO:5, or
(2) a sequence of no more than 500 contiguous nucleotides comprising a sequence having 90% sequence identity to SEQ ID NO: 9, that retains the ability to express a payload sequence in dorsal root ganglion (DRG) neurons or trigeminal ganglia; and
   (b) a payload sequence,
   wherein the promoter of (a) is operably linked to the payload sequence of (b).
   5. The expression construct according to any of 1 to 4, wherein the Avil promoter consists of no more than 490, 480, 470, 460, 450, 440, 430, 420, 410 or 400 nucleotides.
   6. A method of silencing the expression of Nav1.7, comprising the administration of a vector or viral vector of the invention to a patient in need thereof.
   7. A method of silencing the expression of Cav2.2, comprising the administration of a vector or viral vector of the invention to a patient in need thereof.
   8. The vector or the AAV vector according to the invention, further comprising a sequence encoding one or more guide RNAs if the payload sequence comprises SaCas9 dSaCas9-KRAB or dSaCas9-VPR.
   9. The vector or AAV vector according to 8, wherein the one or more guide RNAs are directed to a Nav1.7 gene or Cav2.2 gene.
   10. The vector or AAV vector according to 9, wherein the guide RNAs are one or more of SEQ ID NO: 10, 11, and 12.

### EXAMPLES

### Sequences

Advillin consensus sequence (394 bp)
SEQ ID NO: 1
NNNNNNYCWKYCTCCTGTCATCASYRTAATRYRTYYAYATCTMCAATATG
GCAAATNTCATATCCTYCCWMMNNNNNNNNCTGMATTATTGATGGVCWRN
NTGCACTTTTWAAAAAATCANWTAGAYCAGGGYGTGGARCTGGARTYMAN
NNNNNNRRGARGCCTTTARAAGTCYGCWCTTCTNGTTNYYGCTGTTTTGM
ATAGGMRCAGATAAAGCTTYCNNCTCTGGTTTGAATAAGYCAAGCYCAGK
GCTAGGTTGGCYSTGATTGGYCAGVRCTRRGARAATKHGGTTAWGATGCA
AACMCAAGCAAATATAACCCDGWRTCTVWNNNNNGCRGCCATTRCTRARM
NMAGGCWKCAGGASSKGVHGNNNNNNNNNNNNNNNNNNNNNNNN

Wherein N at position 1 is C or no nucleotide, N at position 2 is C or no nucleotide, N at position 3 is G, A or no nucleotide, N at position 4 is G or no nucleotide, N at position 5 is C or no nucleotide, N at position 6 is C or no nucleotide, N at position 57 is T or no nucleotide, N at position 73 is C or no nucleotide, N at position 74 is T or no nucleotide, N at position 75 is C or no nucleotide, N at position 76 is T or no nucleotide, N at position 77 is T or no nucleotide, N at position 78 is T or no nucleotide, N at position 79 is C or no nucleotide, N at position 80 is C or no nucleotide, N at position 100 is T or no nucleotide, N at position 101 is G or no nucleotide, N at position 121 is A or no nucleotide, N at position 150 is A or no nucleotide, N at position 151 is A or no nucleotide, N at position 152 is G or no nucleotide, N at position 153 is A or no nucleotide, N at position 154 is A or no nucleotide, N at position 155 is A or no nucleotide, N at position 156 is A or no nucleotide, N at position 184 is T or no nucleotide, N at position 188 is T or no nucleotide, N at position 222 is C or no nucleotide, N at position 223 is C, T or no nucleotide, N at position 330 is G or no nucleotide, N at position 331 is C or no nucleotide, N at position 332 is T or no nucleotide, N at position 333 is G or no nucleotide, N at position 334 is T or no nucleotide, N at position 351 is T or no nucleotide, N at position 371 is C or no nucleotide, N at position 372 is C or no nucleotide, N at position 373 is T or no nucleotide, N at position 374 is C or no nucleotide, N at position 375 is C or no nucleotide, N at position 376 is C, T or no nucleotide, N at position 377 is G or no nucleotide, N at position 378 is C or no nucleotide, N at position 379 is C, T or no nucleotide, N at position 380 is C, T or no nucleotide, N at position 381 is C, T or no nucleotide, N at position 382 is G, T or no nucleotide, N at position 383 is G or no nucleotide, N at position 384 is A, G or no nucleotide, N at position 385 is G or no nucleotide, N at position 386 is T or no nucleotide, N at position 387 is G or no nucleotide, N at position 388 is G or no nucleotide, N at position 389 is T or no nucleotide, N at position 390 is T or no nucleotide, N at position 391 is C or no nucleotide, N at position 392 is C, T or no nucleotide, N at position 393 is C, T or no nucleotide and N at position 394 is C, T or no nucleotide.

human Advillin promoter fragment (378 bp)
SEQ ID NO: 2
CCAGCCTCTTCCTCCTGTCATCACCATAATGCATCCATATCTACAATATGGCAAATTTCATATCCTTCCAACCTC
TTTCCTGCATTATTGATGGGCTGTGTGCACTTTTTAAAAAATCAATTAGATCAGGGCGTGGAGCTGGAGTTCAAA
GAAGCCTTTAAAAGTCTGCTCTTCTGTTTTGCTGTTTTGAATAGGCACAGATAAAGCTTTCCCTCTGGTTTGAAT
AAGCCAAGCTCAGTGCTAGGTTGGCTCTGATTGGCCAGGACTAGGAAAATGCGGTTAAGATGCAAACACAAGCAA
ATATAACCCAGTATCTCTGCGGCCATTACTAAGCTAAGGCAGCAGGACCTGGAGCCTCCTGCTTTGGAGTGGTTC
TTC
mouse Advillin promoter fragment (344 bp)
SEQ ID NO: 3
CCAGTCTCCTGTCATCAGCATAATACGTTCATATCTACAATATGGCAAATTCATATCCTCCCTCACTGAATTATT
GATGGACAGTGCACTTTTAAAAAAATCAATAGACCAGGGTGTGGAGCTGGAGTTAAAAGAAGCCTTTAGAAGTCT
GCTCTTCTTGTTTTCGCTGTTTTGCATAGGAGCAGATAAAGCTTTCCTCTCTGGTTTGAATAAGTCAAGCCCAGG
GCTAGGTTGGCTGTGATTGGCCAGAGCTAGGAGAATGTGGTTATGATGCAAACACAAGCAAATATAACCCTGTGT
CTATGCTGTGCGGCCATTGCTGAAACAGGCTTCAGGACGGGCTG
pig Advillin promoter fragment (376 bp)
SEQ ID NO: 4
CCAGCCCCTGCCTCCTGTCATCAGTGTAATGTGTCTACATCTCCAATATGGCAAATTTCATATCCTCCCACCCTC
TTTCTGCATTATTGATGGCCTGTGCACTTTTAAAAAAATCAATTAGATCAGGGCGTGGAGCTGGAGTTAAAAGAA
GCCTTTAAAAGTCTGCACTTCTTGTTTTGCTGTTTTGAATAGGAACAGATAAAGCTTTCCCTCTGGTTTGAATAA
GTCAAGCCCAGGGCTAGGTTGGCTGTGATTGGTCAGGACTAAGAAAATGCGGTTAAGATGCAAACCCAAGCAAAT
ATAACCCAGTGTCTGAGCAGCCATTACTGAGCTAAGGCAGCAGGACCGGAAGCCTCCCGCTTTTGGGTGGTTCCT
C
dog Advillin promoter fragment (386 bp)
SEQ ID NO: 5
CCGGCCCCTGCCTCCTGTCATCAGCATAATGCATCCATATCTACAATATGGCAAATTTCATATCCTCCCACCCTC
TTTCCCTGCATTATTGATGGGCTATGCACTTTTAAAAAAATCAATTAGATCAGGGTGTGGAACTGGAATCAAAAG
AAAAGGGAGGCCTTTAAAAGTCCGCTCTTCTTGTTTCTGCTGTTTTGAATAGGAGCAGATAAAGCTTCCCCTCTG
GTTTGAATAAGTCAAGCCCAGGGCTAGGTTGGCCGTGATTGGCCAGCACTGGGAAAATTAGGTTAAGATGCAAAC
ACAAGCAAATATAACCCGGAGTCTGTGCAGCCATTGCTAAGCTAAGGCAGCAGGAGCTGGCGCCTCCCGCCCCGG
AGTGGTTCTCT
human Advillin larger promoter region (including exon 1)
SEQ ID NO: 6
TGTGAGCTGCCACCAACCTTCATTGGCCCTTAAGTGTTCACTGGGCCCATGGCAAGCACCGCCTTTCTTCTCGCA
ACCCCTTCCATCATGAAGCAGCTGCACCCCCGGGCTGAGTGGGGTGTTGCACCCACCGGGAGGCCAGCTGCAGAT
GCCACACAGGGCTTCCGCTCAAGGTTACACAAGTTCTGCTGTGATCAACCCCATTGCAGTCAGGTGCTTAGCACG
TGTATATGCTTGATGTCCTATTGACTTCTGGGCATGAACCAGAAGCCCTAAGGGAATGGATTATTATTTTTTTTT
TTTTTTTTGAAATGGAGTCTTGCTCTGTTGCCCAGGCTAGAGGCAGTGGCATGATCTCAGCTCACTGCAACCTCC
ACCTCCCAGGTTCAAGCGATTCTCCTGCCTCAGTCTCCTGAGTAGCTGGGATTACAGGCGCCTGCCAGCACGCCC
GGCTCATTTTTGTATTTTTAGTAGAGATGGGGTTTCACCATCTTGGCCAGGCTGTTTTCAAACTCCTCACATTGT
GATCCACCCACCTCAGCCTCCCAAAGTGCTGGGATTACAGGCGTGAGCCACCACCACACCCAGCCCTGGGAATGG
AGTCTTGATTCTTCTCTGCCCCTCACTGATTCTTCCAAACTGGAAACCCTGAAGCTGAGAGCCCAGCATGGTTCC
TGGCAAACAGCAGGCACTCAAATATTGATTGGTTTACTGTATGACTAGTAGAGACCCCAACGAGCAAAACTGTGG
CCTAATAAAATTCTGGCTCCTCTCCCAGACTTCCCCTCCCTTTGAGAAATGCCAGAAGCTTCTTAGGGAGGCTCT
TGCCAACCTAGACATCACAGGCACTCATGGGGCAGCTCCAGCCTCTTCCTCCTGTCATCACCATAATGCATCCAT
ATCTACAATATGGCAAATTTCATATCCTTCCAACCTCTTTCCTGCATTATTGATGGGCTGTGTGCACTTTTTAAA
AAATCAATTAGATCAGGGCGTGGAGCTGGAGTTCAAAGAAGCCTTTAAAAGTCTGCTCTTCTGTTTTGCTGTTTT
GAATAGGCACAGATAAAGCTTTCCCTCTGGTTTGAATAAGCCAAGCTCAGTGCTAGGTTGGCTCTGATTGGCCAG
GACTAGGAAAATGCGGTTAAGATGCAAACACAAGCAAATATAACCCAGTATCTCTGCGGCCATTACTAAGCTAAG
GCAGCAGGACCTGGAGCCTCCTGCTTTGGAGTGGTTCTTCAATACTGCTGCTGCTTACGCGCCGGGAAACTGGGA
AGGCTGGTGAGCGAGAAGGCAAG
mouse Advillin larger promoter region (including exon 1)
SEQ ID NO: 7
ACTGGGAGCTGCTTTTGCTTTCCTTCCCTTCCACTGACCTGACACTCGGTGGACACTGTTCTTCCTGCATGACCT
CGTTGATCGTGAAATGATAGCACCTGGCAGAGCAAACGCTGGGATTCTGATCAAGGTTACAGAGGTTGGAGTGCA
TTAGCCCCTCTCGGGCCTGGGTTGGAACCAACTCCCTCTCCCCAGGTACTTGGCAAGCGTTTGCTTGCTGTGCTA
TTGACTGGGGTAGAGACTGAAGCCCTGGGGGAATTGGAGGCTCCTGCATGTGCTCTTGATGCTGCCAAACAGGCC
TTGAGATTCAGAGCCCAGCATGGTTTCCAGGCACATAGTAGGATGACTTGTACAGACTCCAAGCAACAAAAGCTT
TCCTGACACCACAGAATTGCCCAGAATCATCTCTCAGCCCTGAACGTCCAGGCACACAGTAGGGGAGCTCTAGCC
AGTCTCCTGTCATCAGCATAATACGTTCATATCTACAATATGGCAAATTCATATCCTCCCTCACTGAATTATTGA
TGGACAGTGCACTTTTAAAAAAATCAATAGACCAGGGTGTGGAGCTGGAGTTAAAAGAAGCCTTTAGAAGTCTGC
TCTTCTTGTTTTCGCTGTTTTGCATAGGAGCAGATAAAGCTTTCCTCTCTGGTTTGAATAAGTCAAGCCCAGGGC
TAGGTTGGCTGTGATTGGCCAGAGCTAGGAGAATGTGGTTATGATGCAAACACAAGCAAATATAACCCTGTGTCT
ATGCTGTGCGGCCATTGCTGAAACAGGCTTCAGGACGGGCTGCTTTGGGAGACCTCATCAGTACTTCACCTGCTG
CTTTGCCGGACTTCGGAGGACAGAGGGTCAG
pig Advillin larger promoter region (including exon 1)
SEQ ID NO: 8
ATCCTGCTGTTGTTCATTTGGGCACTTAACCCCTTCTTCCCTCAAAAGCCCTCTGTGGTGATGAATGATGAAGCC
AGAGCGCCCAGGCTGGGAGGCCAGCTACGGACTCAGCACAGGGGTTGTGCTCCAGGTTACAGAAGTCTGGCTATG
TTAGCTCTTCCCTGGCCTCCATGTGGAACCAAGCCCAGTGCCATCCAGGTGCTTAATAAGCATATGCTTGGGGTC
TTAACTGACTTCTAGGGAGGAACCAGAAGCCCCAAGGGACTTGGACTCTTCTTTCTTCTGTGCTCCTCACTGATT
TTCCCAAACTAGGAGCCCTGAAACTCAGCCCAGGGTAGTTCCTGGTACTTAGTAGGCACTGAGATACTGATTGGA
TGGTTTTGCCCAAGGGGCAAAACAGGATGATGTAATAACATACTCCTGGCCCCTCTCCCAGACTGCCCCTCCGAG
AAATGCCCAGCTGGGAGGGGCTTGCCAGCCTGGACATCCCAGGTGCTCAGGGGGCAGCCCCAGCCCCTGCCTCCT
GTCATCAGTGTAATGTGTCTACATCTCCAATATGGCAAATTTCATATCCTCCCACCCTCTTTCTGCATTATTGAT
GGCCTGTGCACTTTTAAAAAAATCAATTAGATCAGGGCGTGGAGCTGGAGTTAAAAGAAGCCTTTAAAAGTCTGC
ACTTCTTGTTTTGCTGTTTTGAATAGGAACAGATAAAGCTTTCCCTCTGGTTTGAATAAGTCAAGCCCAGGGCTA
GGTTGGCTGTGATTGGTCAGGACTAAGAAAATGCGGTTAAGATGCAAACCCAAGCAAATATAACCCAGTGTCTGA
GCAGCCATTACTGAGCTAAGGCAGCAGGACCGGAAGCCTCCCGCTTTTGGGTGGTTCCTCAACACTAATTATCCG
CCAAAAAACTGGGAAGACTGGTGAGAGAGAAAACAAG
dog Advillin larger promoter region (including exon 1)
SEQ ID NO: 9
CCCTGGTGTCGTTCATTCGGGAGTCACCGGCACACTCTTCTTCCCGTGCATCCAACCCCTTGTGTCACGGCAGAG
CTGCGCACCCAGGCTGGGCAGGCTGTTGCTCCGGCCGGGAGGCCAGCTACGGACTCGGCTCGGCTTCCGCTCAAG
GTTACAGAAGTTGCACTACATGAGTTCTGTTCTGGCCCCCCGTGTGGAAGCAGCTCCGTTGCCGTCCAGGTGCTT
GATAAGCATACGCTTGGTGTCCCATTGACTTCTGGGAATGAACCAGGAGCCCCGAGGGAGTAGGAGTCTTCTGTG
CCCCTTACTGCTTCTCCCAGACTGGGATCCCCGGAACCTAGAATCCATGATTCTTGGCACATAGTAGGCACTCAG
ATTCTGGTTGGACTGTTGTATGGACCCCAAGGGAAGCCCTAAAGAGCAAAACAGGATGACCTAATATAACACATC
CTGGCCCCTCCCCTAGACCTCTCTGCCTCTGAGAAATACCCAGAAGCTTCTCAGGGAGGCACTTGCCAGCCCAGG
CACTCAGGGGCAGCTCCGGCCCCTGCCTCCTGTCATCAGCATAATGCATCCATATCTACAATATGGCAAATTTCA
TATCCTCCCACCCTCTTTCCCTGCATTATTGATGGGCTATGCACTTTTAAAAAAATCAATTAGATCAGGGTGTGG
AACTGGAATCAAAAGAAAAGGGAGGCCTTTAAAAGTCCGCTCTTCTTGTTTCTGCTGTTTTGAATAGGAGCAGAT
AAAGCTTCCCCTCTGGTTTGAATAAGTCAAGCCCAGGGCTAGGTTGGCCGTGATTGGCCAGCACTGGGAAAATTA
GGTTAAGATGCAAACACAAGCAAATATAACCCGGAGTCTGTGCAGCCATTGCTAAGCTAAGGCAGCAGGAGCTGG
CGCCTCCCGCCCCGGAGTGGTTCTCTGGATACCGCCTGGAAACCCGGTGGGAGCCCGGCCGGAGAGCAGGCAAG
Scn9a guide sequence 1
SEQ ID NO: 10
CCACTTTATGTGGCTTGAAGG
Scn9a guide sequence 2
SEQ ID NO: 11
AAGGAGAGAGCTGAGTCAGGT
Scn9a guide sequence 3
SEQ ID NO: 12
TCCTCGATGCTCCCTGAGCTC
SaCas9
SEQ ID NO: 13
AAGCGGAACTACATCCTGGGCCTGGACATCGGCATCACCAGCGTGGGCTACGGCATCATCGACTACGAGACACGG
GACGTGATCGATGCCGGCGTGCGGCTGTTCAAAGAGGCCAACGTGGAAAACAACGAGGGCAGGCGGAGCAAGAGA
GGCGCCAGAAGGCTGAAGCGGCGGAGGCGGCATAGAATCCAGAGAGTGAAGAAGCTGCTGTTCGACTACAACCTG
CTGACCGACCACAGCGAGCTGAGCGGCATCAACCCCTACGAGGCCAGAGTGAAGGGCCTGAGCCAGAAGCTGAGC
GAGGAAGAGTTCTCTGCCGCCCTGCTGCACCTGGCCAAGAGAAGAGGCGTGCACAACGTGAACGAGGTGGAAGAG
GACACCGGCAACGAGCTGTCCACCAAAGAGCAGATCAGCCGGAACAGCAAGGCCCTGGAAGAGAAATACGTGGCC
GAACTGCAGCTGGAACGGCTGAAGAAAGACGGCGAAGTGCGGGGCAGCATCAACAGATTCAAGACCAGCGACTAC
GTGAAAGAAGCCAAACAGCTGCTGAAGGTGCAGAAGGCCTACCACCAGCTGGACCAGAGCTTCATCGACACCTAC
ATCGACCTGCTGGAAACCCGGCGGACCTACTATGAGGGACCTGGCGAGGGCAGCCCCTTCGGCTGGAAGGACATC
AAAGAATGGTACGAGATGCTGATGGGCCACTGCACCTACTTCCCCGAGGAACTGCGGAGCGTGAAGTACGCCTAC
AACGCCGACCTGTACAACGCCCTGAACGACCTGAACAATCTCGTGATCACCAGGGACGAGAACGAGAAGCTGGAA
TATTACGAGAAGTTCCAGATCATCGAGAACGTGTTCAAGCAGAAGAAGAAGCCCACCCTGAAGCAGATCGCCAAA
GAAATCCTCGTGAACGAAGAGGATATTAAGGGCTACAGAGTGACCAGCACCGGCAAGCCCGAGTTCACCAACCTG
AAGGTGTACCACGACATCAAGGACATTACCGCCCGGAAAGAGATTATTGAGAACGCCGAGCTGCTGGATCAGATT
GCCAAGATCCTGACCATCTACCAGAGCAGCGAGGACATCCAGGAAGAACTGACCAATCTGAACTCCGAGCTGACC
CAGGAAGAGATCGAGCAGATCTCTAATCTGAAGGGCTATACCGGCACCCACAACCTGAGCCTGAAGGCCATCAAC
CTGATCCTGGACGAGCTGTGGCACACCAACGACAACCAGATCGCTATCTTCAACCGGCTGAAGCTGGTGCCCAAG
AAGGTGGACCTGTCCCAGCAGAAAGAGATCCCCACCACCCTGGTGGACGACTTCATCCTGAGCCCCGTCGTGAAG
AGAAGCTTCATCCAGAGCATCAAAGTGATCAACGCCATCATCAAGAAGTACGGCCTGCCCAACGACATCATTATC
GAGCTGGCCCGCGAGAAGAACTCCAAGGACGCCCAGAAAATGATCAACGAGATGCAGAAGCGGAACCGGCAGACC
AACGAGCGGATCGAGGAAATCATCCGGACCACCGGCAAAGAGAACGCCAAGTACCTGATCGAGAAGATCAAGCTG
CACGACATGCAGGAAGGCAAGTGCCTGTACAGCCTGGAAGCCATCCCTCTGGAAGATCTGCTGAACAACCCCTTC
AACTATGAGGTGGACCACATCATCCCCAGAAGCGTGTCCTTCGACAACAGCTTCAACAACAAGGTGCTCGTGAAG
CAGGAAGAAAACAGCAAGAAGGGCAACCGGACCCCATTCCAGTACCTGAGCAGCAGCGACAGCAAGATCAGCTAC
GAAACCTTCAAGAAGCACATCCTGAATCTGGCCAAGGGCAAGGGCAGAATCAGCAAGACCAAGAAAGAGTATCTG
CTGGAAGAACGGGACATCAACAGGTTCTCCGTGCAGAAAGACTTCATCAACCGGAACCTGGTGGATACCAGATAC
GCCACCAGAGGCCTGATGAACCTGCTGCGGAGCTACTTCAGAGTGAACAACCTGGACGTGAAAGTGAAGTCCATC
AATGGCGGCTTCACCAGCTTTCTGCGGCGGAAGTGGAAGTTTAAGAAAGAGCGGAACAAGGGGTACAAGCACCAC
GCCGAGGACGCCCTGATCATTGCCAACGCCGATTTCATCTTCAAAGAGTGGAAGAAACTGGACAAGGCCAAAAAA
GTGATGGAAAACCAGATGTTCGAGGAAAAGCAGGCCGAGAGCATGCCCGAGATCGAAACCGAGCAGGAGTACAAA
GAGATCTTCATCACCCCCCACCAGATCAAGCACATTAAGGACTTCAAGGACTACAAGTACAGCCACCGGGTGGAC
AAGAAGCCTAATAGAGAGCTGATTAACGACACCCTGTACTCCACCCGGAAGGACGACAAGGGCAACACCCTGATC
GTGAACAATCTGAACGGCCTGTACGACAAGGACAATGACAAGCTGAAAAAGCTGATCAACAAGAGCCCCGAAAAG
CTGCTGATGTACCACCACGACCCCCAGACCTACCAGAAACTGAAGCTGATTATGGAACAGTACGGCGACGAGAAG
AATCCCCTGTACAAGTACTACGAGGAAACCGGGAACTACCTGACCAAGTACTCCAAAAAGGACAACGGCCCCGTG
ATCAAGAAGATTAAGTATTACGGCAACAAACTGAACGCCCATCTGGACATCACCGACGACTACCCCAACAGCAGA
AACAAGGTCGTGAAGCTGTCCCTGAAGCCCTACAGATTCGACGTGTACCTGGACAATGGCGTGTACAAGTTCGTG
ACCGTGAAGAATCTGGATGTGATCAAAAAAGAAAACTACTACGAAGTGAATAGCAAGTGCTATGAGGAAGCTAAG
AAGCTGAAGAAGATCAGCAACCAGGCCGAGTTTATCGCCTCCTTCTACAACAACGATCTGATCAAGATCAACGGC
GAGCTGTATAGAGTGATCGGCGTGAACAACGACCTGCTGAACCGGATCGAAGTGAACATGATCGACATCACCTAC
CGCGAGTACCTGGAAAACATGAACGACAAGAGGCCCCCCAGGATCATTAAGACAATCGCCTCCAAGACCCAGAGC
ATTAAGAAGTACAGCACAGACATTCTGGGCAACCTGTATGAAGTGAAATCTAAGAAGCACCCTCAGATCATCAAA
AAGGGC
dSaCas9
SEQ ID NO: 14
aagcggaactacatcctgggcctggccatcggcatcaccagcgtgggctacggcatcatcgactacgagacacgg
gacgtgatcgatgccggcgtgcggctgttcaaagaggccaacgtggaaaacaacgagggcaggcggagcaagaga
ggcgccagaaggctgaagcggcggaggcggcatagaatccagagagtgaagaagctgctgttcgactacaacctg
ctgaccgaccacagcgagctgagcggcatcaacccctacgaggccagagtgaagggcctgagccagaagctgagc
gaggaagagttctctgccgccctgctgcacctggccaagagaagaggcgtgcacaacgtgaacgaggtggaagag
gacaccggcaacgagctgtccaccaaagagcagatcagccggaacagcaaggccctggaagagaaatacgtggcc
gaactgcagctggaacggctgaagaaagacggcgaagtgcggggcagcatcaacagattcaagaccagcgactac
gtgaaagaagccaaacagctgctgaaggtgcagaaggcctaccaccagctggaccagagcttcatcgacacctac
atcgacctgctggaaacccggcggacctactatgagggacctggcgagggcagccccttcggctggaaggacatc
aaagaatggtacgagatgctgatgggccactgcacctacttccccgaggaactgcggagcgtgaagtacgcctac
aacgccgacctgtacaacgccctgaacgacctgaacaatctcgtgatcaccagggacgagaacgagaagctggaa
tattacgagaagttccagatcatcgagaacgtgttcaagcagaagaagaagcccaccctgaagcagatcgccaaa
gaaatcctcgtgaacgaagaggatattaagggctacagagtgaccagcaccggcaagcccgagttcaccaacctg
aaggtgtaccacgacatcaaggacattaccgcccggaaagagattattgagaacgccgagctgctggatcagatt
gccaagatcctgaccatctaccagagcagcgaggacatccaggaagaactgaccaatctgaactccgagctgacc
caggaagagatcgagcagatctctaatctgaagggctataccggcacccacaacctgagcctgaaggccatcaac
ctgatcctggacgagctgtggcacaccaacgacaaccagatcgctatcttcaaccggctgaagctggtgcccaag
aaggtggacctgtcccagcagaaagagatccccaccaccctggtggacgacttcatcctgagccccgtcgtgaag
agaagcttcatccagagcatcaaagtgatcaacgccatcatcaagaagtacggcctgcccaacgacatcattatc
gagctggcccgcgagaagaactccaaggacgcccagaaaatgatcaacgagatgcagaagcggaaccggcagacc
aacgagcggatcgaggaaatcatccggaccaccggcaaagagaacgccaagtacctgatcgagaagatcaagctg
cacgacatgcaggaaggcaagtgcctgtacagcctggaagccatccctctggaagatctgctgaacaaccccttc
aactatgaggtggaccacatcatccccagaagcgtgtccttcgacaacagcttcaacaacaaggtgctcgtgaag
caggaagaagccagcaagaagggcaaccggaccccattccagtacctgagcagcagcgacagcaagatcagctac
gaaaccttcaagaagcacatcctgaatctggccaagggcaagggcagaatcagcaagaccaagaaagagtatctg
ctggaagaacgggacatcaacaggttctccgtgcagaaagacttcatcaaccggaacctggtggataccagatac
gccaccagaggcctgatgaacctgctgcggagctacttcagagtgaacaacctggacgtgaaagtgaagtccatc
aatggcggcttcaccagctttctgcggcggaagtggaagtttaagaaagagcggaacaaggggtacaagcaccac
gccgaggacgccctgatcattgccaacgccgatttcatcttcaaagagtggaagaaactggacaaggccaaaaaa
gtgatggaaaaccagatgttcgaggaaaagcaggccgagagcatgcccgagatcgaaaccgagcaggagtacaaa
gagatcttcatcaccccccaccagatcaagcacattaaggacttcaaggactacaagtacagccaccgggtggac
aagaagcctaatagagagctgattaacgacaccctgtactccacccggaaggacgacaagggcaacaccctgatc
gtgaacaatctgaacggcctgtacgacaaggacaatgacaagctgaaaaagctgatcaacaagagccccgaaaag
ctgctgatgtaccaccacgacccccagacctaccagaaactgaagctgattatggaacagtacggcgacgagaag
aatcccctgtacaagtactacgaggaaaccgggaactacctgaccaagtactccaaaaaggacaacggccccgtg
atcaagaagattaagtattacggcaacaaactgaacgcccatctggacatcaccgacgactaccccaacagcaga
aacaaggtcgtgaagctgtccctgaagccctacagattcgacgtgtacctggacaatggcgtgtacaagttcgtg
accgtgaagaatctggatgtgatcaaaaaagaaaactactacgaagtgaatagcaagtgctatgaggaagctaag
aagctgaagaagatcagcaaccaggccgagtttatcgcctccttctacaacaacgatctgatcaagatcaacggc
gagctgtatagagtgatcggcgtgaacaacgacctgctgaaccggatcgaagtgaacatgatcgacatcacctac
cgcgagtacctggaaaacatgaacgacaagaggccccccaggatcattaagacaatcgcctccaagacccagagc
attaagaagtacagcacagacattctgggcaacctgtatgaagtgaaatctaagaagcaccctcagatcatcaaa
aagggc
KRAB domain
SEQ ID NO: 15
CGGACACTGGTGACCTTCAAGGATGTGTTTGTGGACTTCACCAGGGAGGAGTGGAAGCTGCTGGACACTGCTCAG
CAGATCCTGTACAGAAATGTGATGCTGGAGAACTATAAGAACCTGGTTTCCTTGGGTTATCAGCTTACTAAGCCA
GATGTGATCCTCCGGTTGGAGAAGGGAGAAGAGCCCTGGCTGGTG
NLS-SaCas9-NLS-3xHA
SEQ ID NO: 16
ATGGCCCCAAAGAAGAAGCGGAAGGTCGGTATCCACGGAGTCCCAGCAGCCAAGCGGAACTACATCCTGGGCCTG
GACATCGGCATCACCAGCGTGGGCTACGGCATCATCGACTACGAGACACGGGACGTGATCGATGCCGGCGTGCGG
CTGTTCAAAGAGGCCAACGTGGAAAACAACGAGGGCAGGCGGAGCAAGAGAGGCGCCAGAAGGCTGAAGCGGCGG
AGGCGGCATAGAATCCAGAGAGTGAAGAAGCTGCTGTTCGACTACAACCTGCTGACCGACCACAGCGAGCTGAGC
GGCATCAACCCCTACGAGGCCAGAGTGAAGGGCCTGAGCCAGAAGCTGAGCGAGGAAGAGTTCTCTGCCGCCCTG
CTGCACCTGGCCAAGAGAAGAGGCGTGCACAACGTGAACGAGGTGGAAGAGGACACCGGCAACGAGCTGTCCACC
AAAGAGCAGATCAGCCGGAACAGCAAGGCCCTGGAAGAGAAATACGTGGCCGAACTGCAGCTGGAACGGCTGAAG
AAAGACGGCGAAGTGCGGGGCAGCATCAACAGATTCAAGACCAGCGACTACGTGAAAGAAGCCAAACAGCTGCTG
AAGGTGCAGAAGGCCTACCACCAGCTGGACCAGAGCTTCATCGACACCTACATCGACCTGCTGGAAACCCGGCGG
ACCTACTATGAGGGACCTGGCGAGGGCAGCCCCTTCGGCTGGAAGGACATCAAAGAATGGTACGAGATGCTGATG
GGCCACTGCACCTACTTCCCCGAGGAACTGCGGAGCGTGAAGTACGCCTACAACGCCGACCTGTACAACGCCCTG
AACGACCTGAACAATCTCGTGATCACCAGGGACGAGAACGAGAAGCTGGAATATTACGAGAAGTTCCAGATCATC
GAGAACGTGTTCAAGCAGAAGAAGAAGCCCACCCTGAAGCAGATCGCCAAAGAAATCCTCGTGAACGAAGAGGAT
ATTAAGGGCTACAGAGTGACCAGCACCGGCAAGCCCGAGTTCACCAACCTGAAGGTGTACCACGACATCAAGGAC
ATTACCGCCCGGAAAGAGATTATTGAGAACGCCGAGCTGCTGGATCAGATTGCCAAGATCCTGACCATCTACCAG
AGCAGCGAGGACATCCAGGAAGAACTGACCAATCTGAACTCCGAGCTGACCCAGGAAGAGATCGAGCAGATCTCT
AATCTGAAGGGCTATACCGGCACCCACAACCTGAGCCTGAAGGCCATCAACCTGATCCTGGACGAGCTGTGGCAC
ACCAACGACAACCAGATCGCTATCTTCAACCGGCTGAAGCTGGTGCCCAAGAAGGTGGACCTGTCCCAGCAGAAA
GAGATCCCCACCACCCTGGTGGACGACTTCATCCTGAGCCCCGTCGTGAAGAGAAGCTTCATCCAGAGCATCAAA
GTGATCAACGCCATCATCAAGAAGTACGGCCTGCCCAACGACATCATTATCGAGCTGGCCCGCGAGAAGAACTCC
AAGGACGCCCAGAAAATGATCAACGAGATGCAGAAGCGGAACCGGCAGACCAACGAGCGGATCGAGGAAATCATC
CGGACCACCGGCAAAGAGAACGCCAAGTACCTGATCGAGAAGATCAAGCTGCACGACATGCAGGAAGGCAAGTGC
CTGTACAGCCTGGAAGCCATCCCTCTGGAAGATCTGCTGAACAACCCCTTCAACTATGAGGTGGACCACATCATC
CCCAGAAGCGTGTCCTTCGACAACAGCTTCAACAACAAGGTGCTCGTGAAGCAGGAAGAAAACAGCAAGAAGGGC
AACCGGACCCCATTCCAGTACCTGAGCAGCAGCGACAGCAAGATCAGCTACGAAACCTTCAAGAAGCACATCCTG
AATGTGGCCAAGGGCAAGGGCAGAATCAGCAAGACCAAGAAAGAGTATCTGCTGGAAGAACGGGACATCAACAGG
TTCTCCGTGCAGAAAGACTTCATCAACCGGAACCTGGTGGATACCAGATACGCCACCAGAGGCCTGATGAACCTG
CTGCGGAGCTACTTCAGAGTGAACAACCTGGACGTGAAAGTGAAGTCCATCAATGGCGGCTTCACCAGCTTTCTG
CGGCGGAAGTGGAAGTTTAAGAAAGAGCGGAACAAGGGGTACAAGCACCACGCCGAGGACGCCCTGATCATTGCC
AACGCCGATTTCATCTTCAAAGAGTGGAAGAAACTGGACAAGGCCAAAAAAGTGATGGAAAACCAGATGTTCGAG
GAAAAGCAGGCCGAGAGCATGCCCGAGATCGAAACCGAGCAGGAGTACAAAGAGATCTTCATCACCCCCCACCAG
ATCAAGCACATTAAGGACTTCAAGGACTACAAGTACAGCCACCGGGTGGACAAGAAGCCTAATAGAGAGCTGATT
AACGACACCCTGTACTCCACCCGGAAGGACGACAAGGGCAACACCCTGATCGTGAACAATCTGAACGGCCTGTAC
GACAAGGACAATGACAAGCTGAAAAAGCTGATCAACAAGAGCCCCGAAAAGCTGCTGATGTACCACCACGACCCC
CAGACCTACCAGAAACTGAAGCTGATTATGGAACAGTACGGCGACGAGAAGAATCCCCTGTACAAGTACTACGAG
GAAACCGGGAACTACCTGACCAAGTACTCCAAAAAGGACAACGGCCCCGTGATCAAGAAGATTAAGTATTACGGC
AACAAACTGAACGCCCATCTGGACATCACCGACGACTACCCCAACAGCAGAAACAAGGTCGTGAAGCTGTCCCTG
AAGCCCTACAGATTCGACGTGTACCTGGACAATGGCGTGTACAAGTTCGTGACCGTGAAGAATCTGGATGTGATC
AAAAAAGAAAACTACTACGAAGTGAATAGCAAGTGCTATGAGGAAGCTAAGAAGCTGAAGAAGATCAGCAACCAG
GCCGAGTTTATCGCCTCCTTCTACAACAACGATCTGATCAAGATCAACGGCGAGCTGTATAGAGTGATCGGCGTG
AACAACGACCTGCTGAACCGGATCGAAGTGAACATGATCGACATCACCTACCGCGAGTACCTGGAAAACATGAAC
GACAAGAGGCCCCCCAGGATCATTAAGACAATCGCCTCCAAGACCCAGAGCATTAAGAAGTACAGCACAGACATT
CTGGGCAACCTGTATGAAGTGAAATCTAAGAAGCACCCTCAGATCATCAAAAAGGGCAAAAGGCCGGCGGCCACG
AAAAAGGCCGGCCAGGCAAAAAAGAAAAAGGGATCCTACCCATACGATGTTCCAGATTACGCTTACCCATACGAT
GTTCCAGATTACGCTTACCCATACGATGTTCCAGATTACGCTTAA
HA-NLS-dSaCas9-NLS-KRAB-NLS
SEQ ID NO: 17
ATGTACCCATACGATGTTCCAGATTACGCTGCCCCAAAGAAGAAGCGGAAGGTCGGTATCCACGGAGTCCCAGCA
GCCAAGCGGAACTACATCCTGGGCCTGGCCATCGGCATCACCAGCGTGGGCTACGGCATCATCGACTACGAGACA
CGGGACGTGATCGATGCCGGCGTGCGGCTGTTCAAAGAGGCCAACGTGGAAAACAACGAGGGCAGGCGGAGCAAG
AGAGGCGCCAGAAGGCTGAAGCGGCGGAGGCGGCATAGAATCCAGAGAGTGAAGAAGCTGCTGTTCGACTACAAC
CTGCTGACCGACCACAGCGAGCTGAGCGGCATCAACCCCTACGAGGCCAGAGTGAAGGGCCTGAGCCAGAAGCTG
AGCGAGGAAGAGTTCTCTGCCGCCCTGCTGCACCTGGCCAAGAGAAGAGGCGTGCACAACGTGAACGAGGTGGAA
GAGGACACCGGCAACGAGCTGTCCACCAAAGAGCAGATCAGCCGGAACAGCAAGGCCCTGGAAGAGAAATACGTG
GCCGAACTGCAGCTGGAACGGCTGAAGAAAGACGGCGAAGTGCGGGGCAGCATCAACAGATTCAAGACCAGCGAC
TACGTGAAAGAAGCCAAACAGCTGCTGAAGGTGCAGAAGGCCTACCACCAGCTGGACCAGAGCTTCATCGACACC
TACATCGACCTGCTGGAAACCCGGCGGACCTACTATGAGGGACCTGGCGAGGGCAGCCCCTTCGGCTGGAAGGAC
ATCAAAGAATGGTACGAGATGCTGATGGGCCACTGCACCTACTTCCCCGAGGAACTGCGGAGCGTGAAGTACGCC
TACAACGCCGACCTGTACAACGCCCTGAACGACCTGAACAATCTCGTGATCACCAGGGACGAGAACGAGAAGCTG
GAATATTACGAGAAGTTCCAGATCATCGAGAACGTGTTCAAGCAGAAGAAGAAGCCCACCCTGAAGCAGATCGCC
AAAGAAATCCTCGTGAACGAAGAGGATATTAAGGGCTACAGAGTGACCAGCACCGGCAAGCCCGAGTTCACCAAC
CTGAAGGTGTACCACGACATCAAGGACATTACCGCCCGGAAAGAGATTATTGAGAACGCCGAGCTGCTGGATCAG
ATTGCCAAGATCCTGACCATCTACCAGAGCAGCGAGGACATCCAGGAAGAACTGACCAATCTGAACTCCGAGCTG
ACCCAGGAAGAGATCGAGCAGATCTCTAATCTGAAGGGCTATACCGGCACCCACAACCTGAGCCTGAAGGCCATC
AACCTGATCCTGGACGAGCTGTGGCACACCAACGACAACCAGATCGCTATCTTCAACCGGCTGAAGCTGGTGCCC
AAGAAGGTGGACCTGTCCCAGCAGAAAGAGATCCCCACCACCCTGGTGGACGACTTCATCCTGAGCCCCGTCGTG
AAGAGAAGCTTCATCCAGAGCATCAAAGTGATCAACGCCATCATCAAGAAGTACGGCCTGCCCAACGACATCATT
ATCGAGCTGGCCCGCGAGAAGAACTCCAAGGACGCCCAGAAAATGATCAACGAGATGCAGAAGCGGAACCGGCAG
ACCAACGAGCGGATCGAGGAAATCATCCGGACCACCGGCAAAGAGAACGCCAAGTACCTGATCGAGAAGATCAAG
CTGCACGACATGCAGGAAGGCAAGTGCCTGTACAGCCTGGAAGCCATCCCTCTGGAAGATCTGCTGAACAACCCC
TTCAACTATGAGGTGGACCACATCATCCCCAGAAGCGTGTCCTTCGACAACAGCTTCAACAACAAGGTGCTCGTG
AAGCAGGAAGAAGCCAGCAAGAAGGGCAACCGGACCCCATTCCAGTACCTGAGCAGCAGCGACAGCAAGATCAGC
TACGAAACCTTCAAGAAGCACATCCTGAATCTGGCCAAGGGCAAGGGCAGAATCAGCAAGACCAAGAAAGAGTAT
CTGCTGGAAGAACGGGACATCAACAGGTTCTCCGTGCAGAAAGACTTCATCAACCGGAACCTGGTGGATACCAGA
TACGCCACCAGAGGCCTGATGAACCTGCTGCGGAGCTACTTCAGAGTGAACAACCTGGACGTGAAAGTGAAGTCC
ATCAATGGCGGCTTCACCAGCTTTCTGCGGCGGAAGTGGAAGTTTAAGAAAGAGCGGAACAAGGGGTACAAGCAC
CACGCCGAGGACGCCCTGATCATTGCCAACGCCGATTTCATCTTCAAAGAGTGGAAGAAACTGGACAAGGCCAAA
AAAGTGATGGAAAACCAGATGTTCGAGGAAAAGCAGGCCGAGAGCATGCCCGAGATCGAAACCGAGCAGGAGTAC
AAAGAGATCTTCATCACCCCCCACCAGATCAAGCACATTAAGGACTTCAAGGACTACAAGTACAGCCACCGGGTG
GACAAGAAGCCTAATAGAGAGCTGATTAACGACACCCTGTACTCCACCCGGAAGGACGACAAGGGCAACACCCTG
ATCGTGAACAATCTGAACGGCCTGTACGACAAGGACAATGACAAGCTGAAAAAGCTGATCAACAAGAGCCCCGAA
AAGCTGCTGATGTACCACCACGACCCCCAGACCTACCAGAAACTGAAGCTGATTATGGAACAGTACGGCGACGAG
AAGAATCCCCTGTACAAGTACTACGAGGAAACCGGGAACTACCTGACCAAGTACTCCAAAAAGGACAACGGCCCC
GTGATCAAGAAGATTAAGTATTACGGCAACAAACTGAACGCCCATCTGGACATCACCGACGACTACCCCAACAGC
AGAAACAAGGTCGTGAAGCTGTCCCTGAAGCCCTACAGATTCGACGTGTACCTGGACAATGGCGTGTACAAGTTC
GTGACCGTGAAGAATCTGGATGTGATCAAAAAAGAAAACTACTACGAAGTGAATAGCAAGTGCTATGAGGAAGCT
AAGAAGCTGAAGAAGATCAGCAACCAGGCCGAGTTTATCGCCTCCTTCTACAACAACGATCTGATCAAGATCAAC
GGCGAGCTGTATAGAGTGATCGGCGTGAACAACGACCTGCTGAACCGGATCGAAGTGAACATGATCGACATCACC
TACCGCGAGTACCTGGAAAACATGAACGACAAGAGGCCCCCCAGGATCATTAAGACAATCGCCTCCAAGACCCAG
AGCATTAAGAAGTACAGCACAGACATTCTGGGCAACCTGTATGAAGTGAAATCTAAGAAGCACCCTCAGATCATC
AAAAAGGGCAAAAGGCCGGCGGCCACGAAAAAGGCCGGCCAGGCAAAAAAGAAAAAGGGATCCGATGCTAAGTCA
CTGACTGCCTGGTCCCGGACACTGGTGACCTTCAAGGATGTGTTTGTGGACTTCACCAGGGAGGAGTGGAAGCTG
CTGGACACTGCTCAGCAGATCCTGTACAGAAATGTGATGCTGGAGAACTATAAGAACCTGGTTTCCTTGGGTTAT
CAGCTTACTAAGCCAGATGTGATCCTCCGGTTGGAGAAGGGAGAAGAGCCCTGGCTGGTGGAGAGAGAAATTCAC
CAAGAGACCCATCCTGATTCAGAGACTGCATTTGAAATCAAATCATCAGTTCCGAAAAAGAAACGCAAAGTTTAA
Human EFS promoter sequence
SEQ ID NO: 18
GGCTCCGGTGCCCGTCAGTGGGCAGAGCGCACATCGCCCACAGTCCCCGAGAAGTTGGGGGGAGGGGTCGGCAAT
TGATCCGGTGCCTAGAGAAGGTGGCGCGGGGTAAACTGGGAAAGTGATGTCGTGTACTGGCTCCGCCTTTTTCCC
GAGGGTGGGGGAGAACCGTATATAAGTGCAGTAGTCGCCGTGAACGTTCTTTTTCGCAACGGGTTTGCCGCCAGA
ACACAGG
Mouse EFS promoter sequence
SEQ ID NO: 19
ACCGGGGAGCTGGCGCGGGGCAAACTGGGAAAGCGGTGTCGTGTGCTGGCTCCGCCCTCTTCCCGAGGGTGGGGG
AGAACGGTATATAAGTGCGGCAGTCGCCTTGGACGTTCTTTTTCGCAACGGGTTTGCCGTCAGAACGCAG

### Example 1: Discovery of Avil Promoter Consensus Sequence

To identify conserved gene regulatory elements in the Avil gene region, a comparative genomic analysis across 100 vertebrate species was performed. Expressed sequence tags (ESTs) were mapped to the 5′ end of the mouse and human Advillin gene sequences in order to help locate the transcriptional start site (TSS) for each gene. Sequence conservation analyses were carried out by analysing ˜1000 bp 5′ and 3′ of the TSS. Conserved sequences were identified in human (SEQ ID NO: 2), mouse (SEQ ID NO: 3), pig (SEQ ID NO: 4) and dog (SEQ ID NO: 5). Surprisingly, the conserved sequences discovered were less than 400 bp in length, indicating that they would be suitable to be used in gene therapy vectors to express payloads in target cells.

The sequences were aligned and the alignment used to create the Avil promoter consensus sequence of SEQ ID NO: 1.

### Example 2: Cloning of Short Avil Promoter

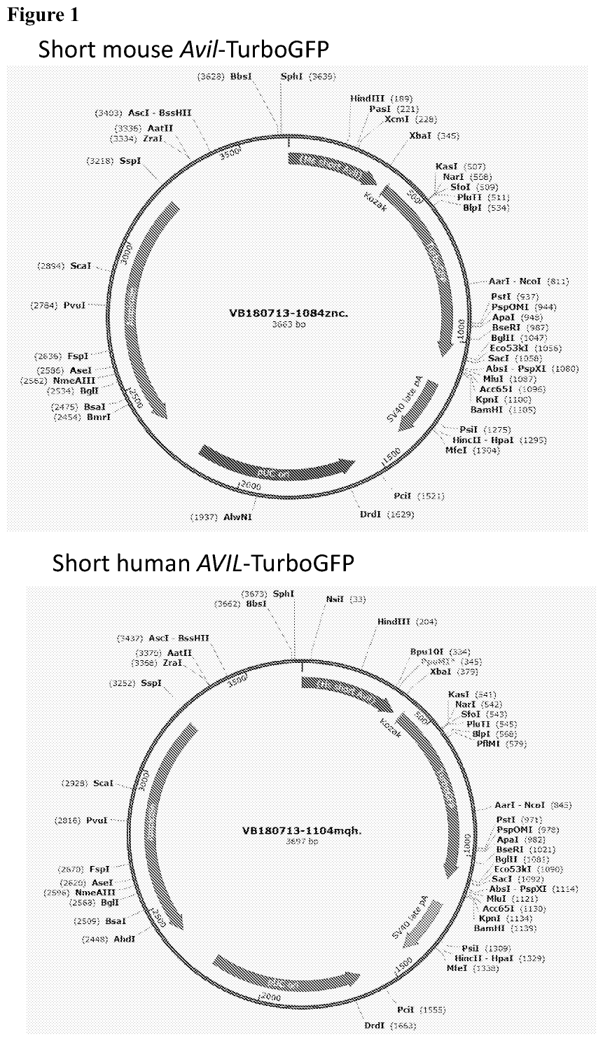

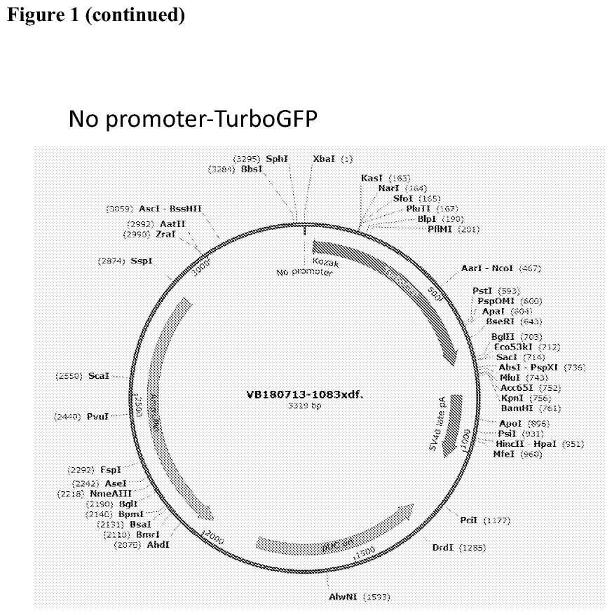

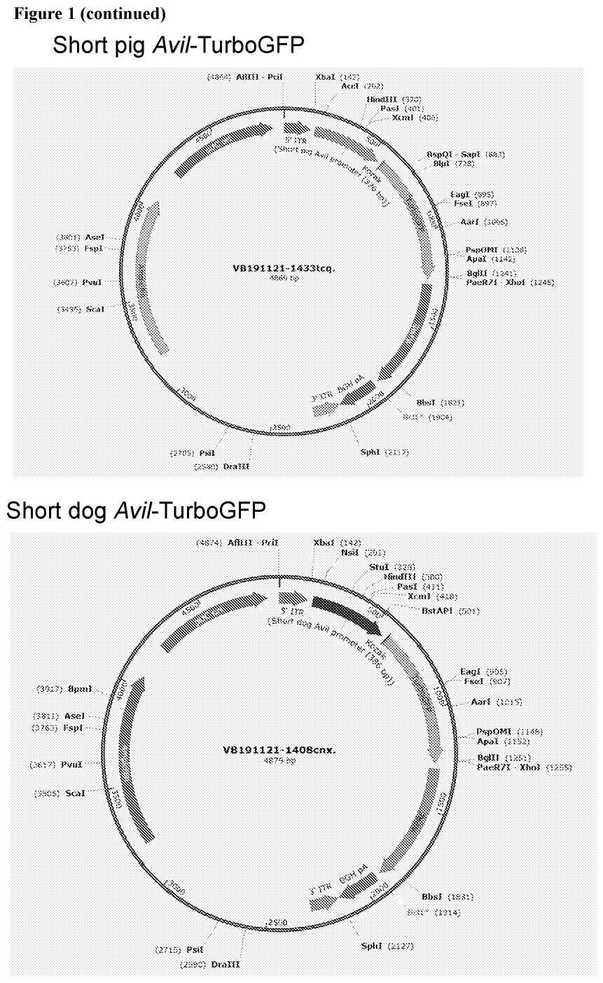

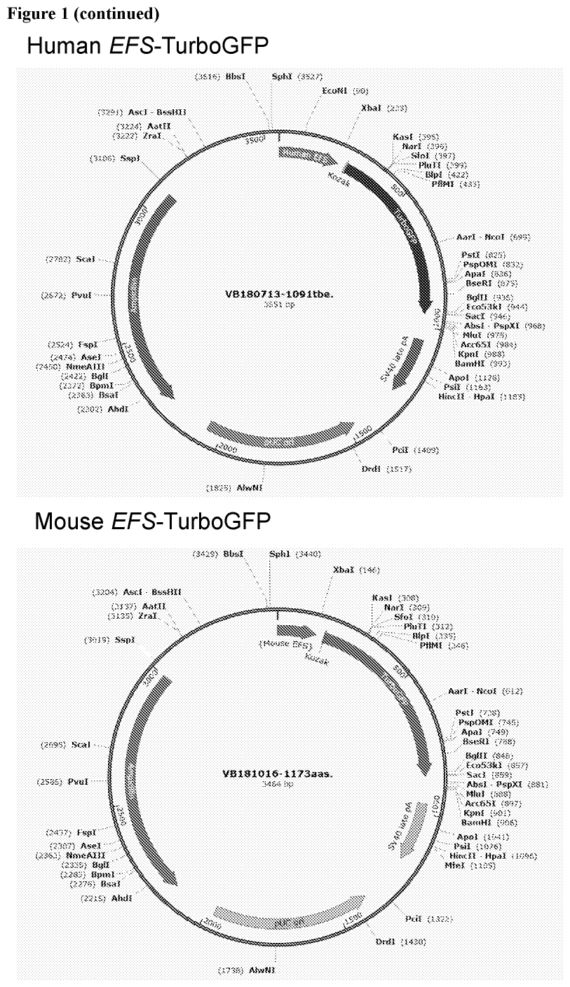

The short mouse Avil promoter (SEQ ID NO: 3) and short human Avil promoter (SEQ ID NO: 2) were cloned upstream of a TurboGFP sequence to give plasmids VB180713-1084znc and VB180713-1104mqh (VectorBuilder) (FIG. 1). The short pig Avil promoter (SEQ ID NO: 4) and short dog Avil promoter (SEQ ID NO: 5) were cloned upstream of a Turbo GFP sequence to give plasmids VB191121-1433tcq and VB191121-1408cnx. As a negative control, the Turbo-GFP plasmid backbone was used but with no promoter sequence cloned upstream (VB180713-1083xdf). As positive controls, the human EFS promoter (SEQ ID NO: 18) and mouse EFS promoter (SEQ ID NO: 19) were cloned upstream of Turbo GFP sequence to give plasmids VB180713-1091tbe and VB181016-1173aas.

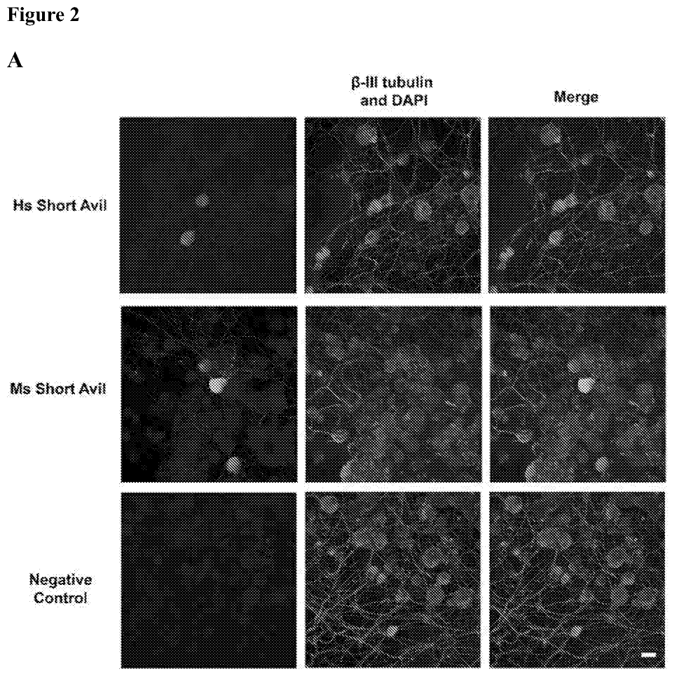

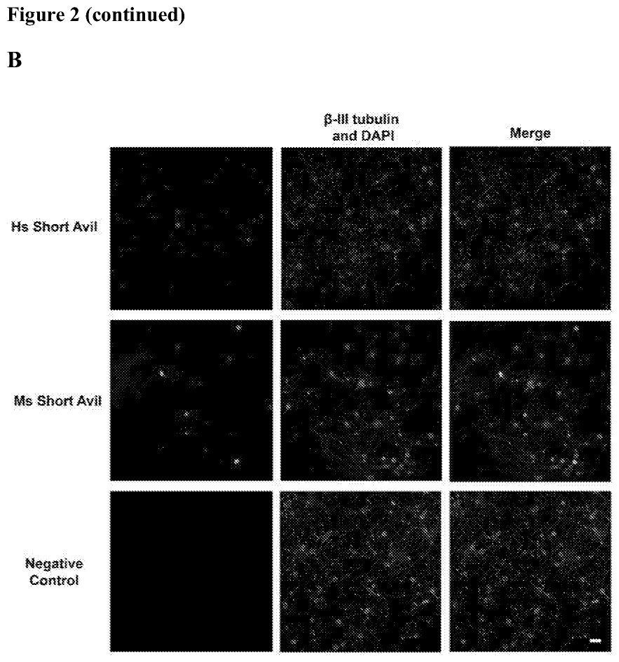

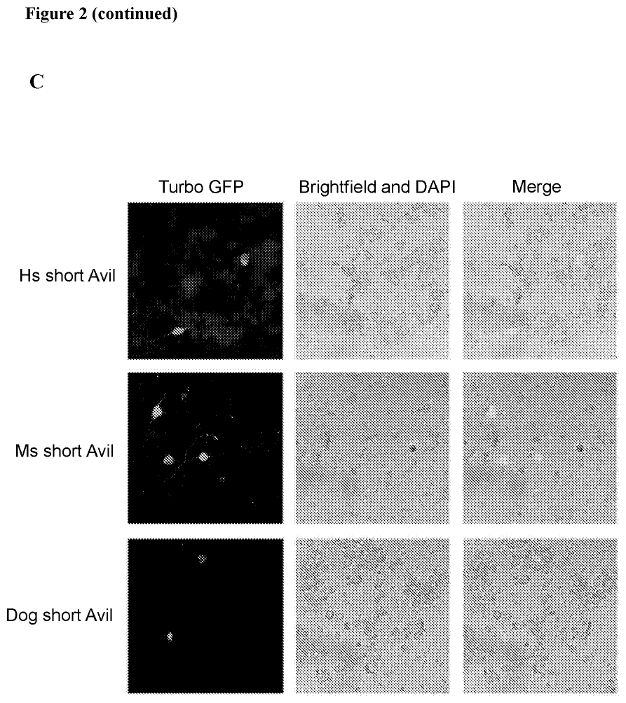

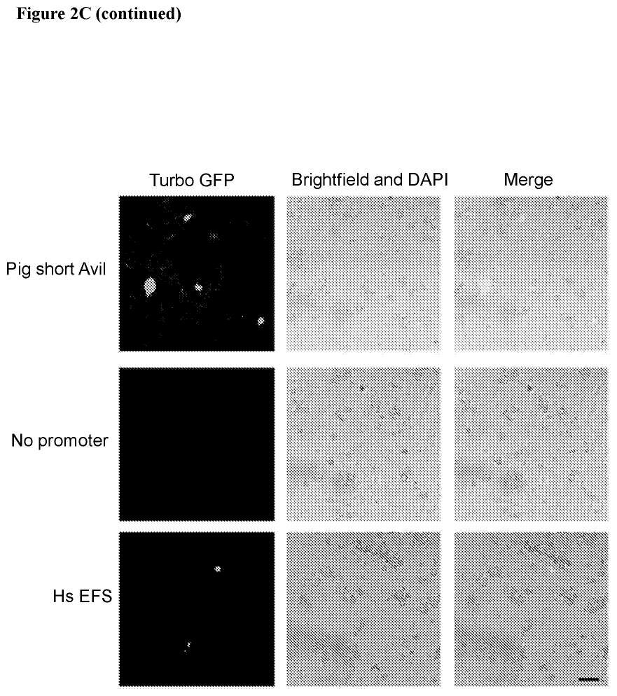

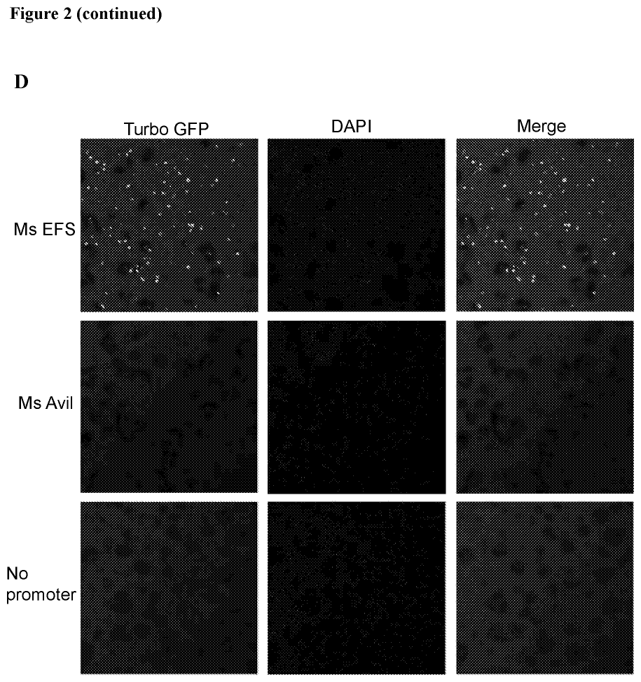

Mouse DRG neurons from adult C57BL6/J wild-type mice were extracted and dissociated as previously described (Emery et al. 2016) and electroporated using 1-2 μg of plasmid DNA using the Lonza 4D-Nucleofector (following the manufacturer's standard protocol). Cells were plated on poly-L-lysine and laminin coated glass coverslips (13 mm; size 0) and cultured in standard cell medium (Dulbecco's modified Eagle medium+GlutaMAX™, Life Technologies) supplemented with 10% foetal bovine serum and nerve growth factor (50 ng/ml). After 48 and 72 hrs the cells were PFA fixed, stained using DAPI and a beta-3 tubulin antibody (ab78078), processed using standard immunocytochemistry protocols and imaged using a Leica TCS SP8 confocal microscope. TurboGFP was detected in beta-3 tubulin-positive transfected DRG neurons for the human and mouse short Avil (FIGS. 2A and B) constructs whilst no TurboGFP positive neurons were detected with the no promoter plasmid control. TurboGFP was also detected in transfected mouse DRG neurons for the pig and dog short Avil constructs (FIG. 2C).

Specificity of expression was also tested in a non-dorsal root ganglia mouse cell line—i.e. CAD (Cath.-a-differentiated) cells which are a variant of a CNS catecholaminergic cell line established from a brain tumour. CAD cells (ECACC) were maintained in DMEM:HAMS F12 (1:1), 2 mM Glutamine and 8% Foetal Bovine Serum (FBS). Cells were seeded in 48 well plates and transfected with 0.5 μg of plasmid DNA using Lipofectamine 3000 (ThermoFisher) according to the manufacturer's conditions. Following transfection, cells were split and re-seeded onto poly-L-lysine coated glass coverslips. After 72 hours, cells were PFA fixed, stained using DAPI and imaged using a Leica TCS SP8 confocal microscope. CAD cells transfected with the mouse EFS promoter plasmid were positive for TurboGFP fluorescence whereas the mouse short Avil and no promoter-TurboGFP transfected cells did not express TurboGFP, highlighting the specificity of expression for the mouse short Avil promoter sequence (FIG. 2D).

### Example 3: Short Mouse Avil Promoter Driving Cre Recombinase in AAV9

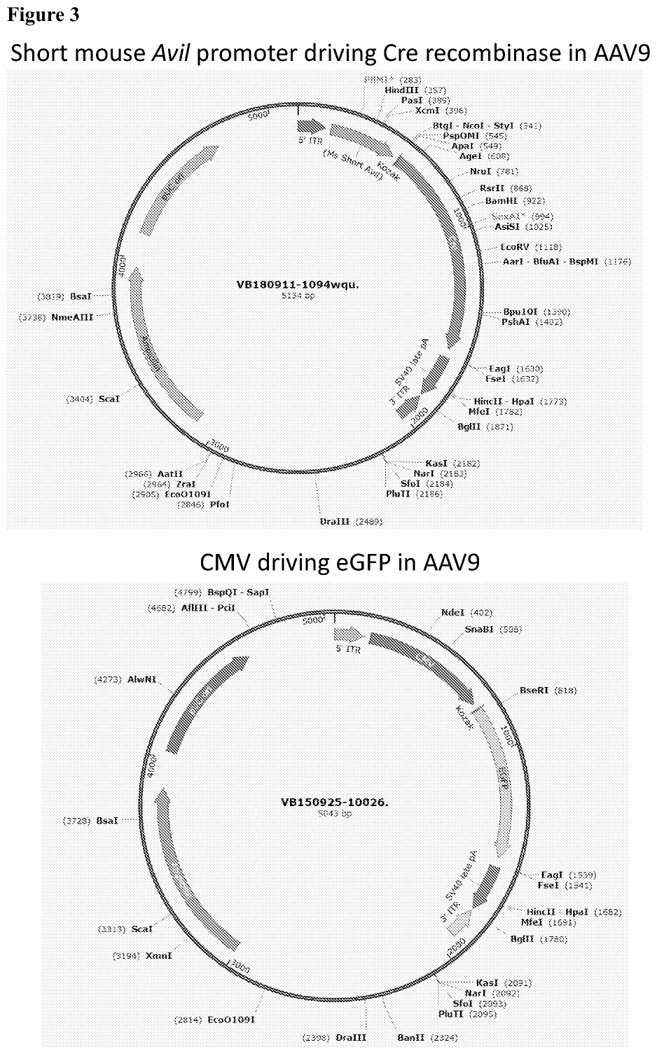

The short mouse Avil promoter (SEQ ID NO: 3) was cloned upstream of Cre recombinase and flanked by AAV inverted terminal repeats to give plasmid VB180911-1094wqu (VectorBuilder). As a control, CMV driving eGFP was cloned between inverted terminal repeats to give plasmid VB150925-10026 (FIG. 3). The cargoes were packaged into AAV9 (VectorBuilder) with titres measured at 2.13×10¹² GC/ml and 2.11×10¹² GC/ml respectively. Intrathecal injections were performed under 2.5% isoflurane anaesthesia and aseptic conditions. First, heterozygous CAG floxed stop tdTomato adult mice (B6; 129S6-Gt(ROSA)26Sor^{tm9(CAG-tdTomato)Hze/}J; Jackson Laboratories stock number 007905) were injected with 200 μl 25% mannitol in PBS to the tail vein. Twenty minutes later, 5 μl of AAV9 virus was injected intrathecally by direct lumbar puncture. 48 hours later, intrathecal injections were repeated in the same way. Intraperitoneal injections of the virus were carried out in ˜P7 heterozygous CAG foxed stop tdTomato mouse pups using a single injection containing 5 μl of AAV9 virus. Seven weeks later (intrathecal-injected mice) or twelve weeks later (following intraperitoneal injections), mice were overdosed by intraperitoneal injection of 20% pentobarbitone sodium and transcardially perfused with PFA. Dissected DRGs (L5/L6) and hearts were fixed in 4% filtered PFA for an hour at 4° C. followed by overnight incubation in 30% sucrose. Tissues were inserted in cryomold filled with mounting medium O.C.T. (Tissue-Tek) and sectioned with a cryostat (Bright OFT5000). DRGs and the heart (transverse sections) were cut at 10 μM per section and mounted on Superfrost Plus glass slides. Following drying, the sections were stained with DAPI and imaged using a Leica TCS SP8 confocal microscope.

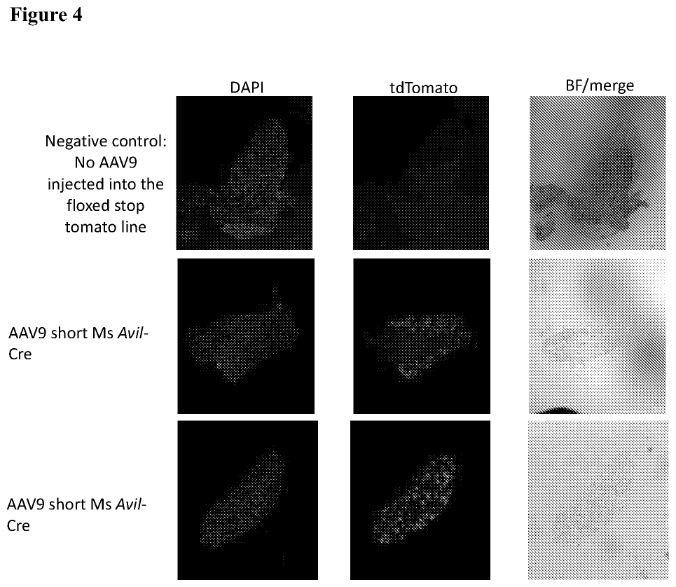

DRG neurons transduced with the short mouse Avil-Cre AAV9 showed clear tdTomato expression in L5 and L6 ganglia (FIGS. 4 and 5). Following IP delivery of virus, the heart had a limited number of cells that were positive for tdTomato in mice injected with AAV9 short mouse Avil-Cre recombinase. In comparison, numerous heart cells expressed eGFP following injection with AAV9 CMV-eGFP (FIG. 6).

### Example 4: Short Mouse Avil Promoter Driving SaCas9 or dSaCas9-KRAB in AAV1

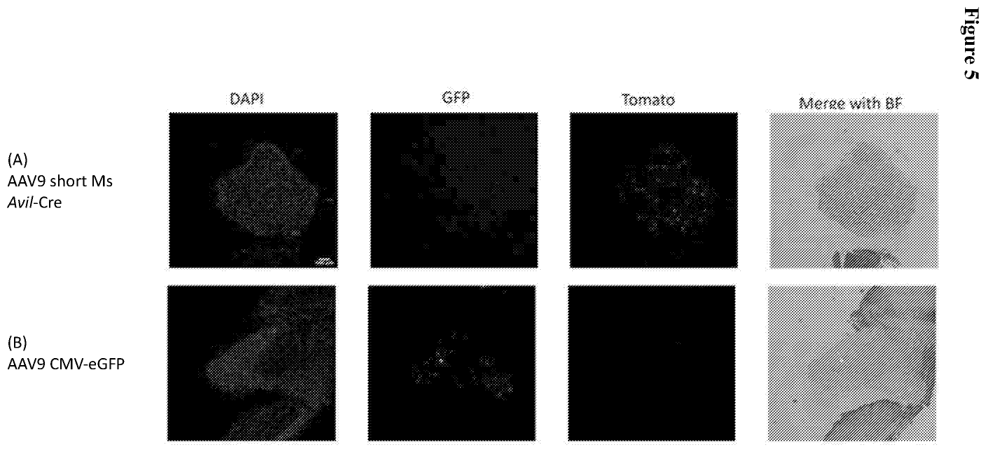

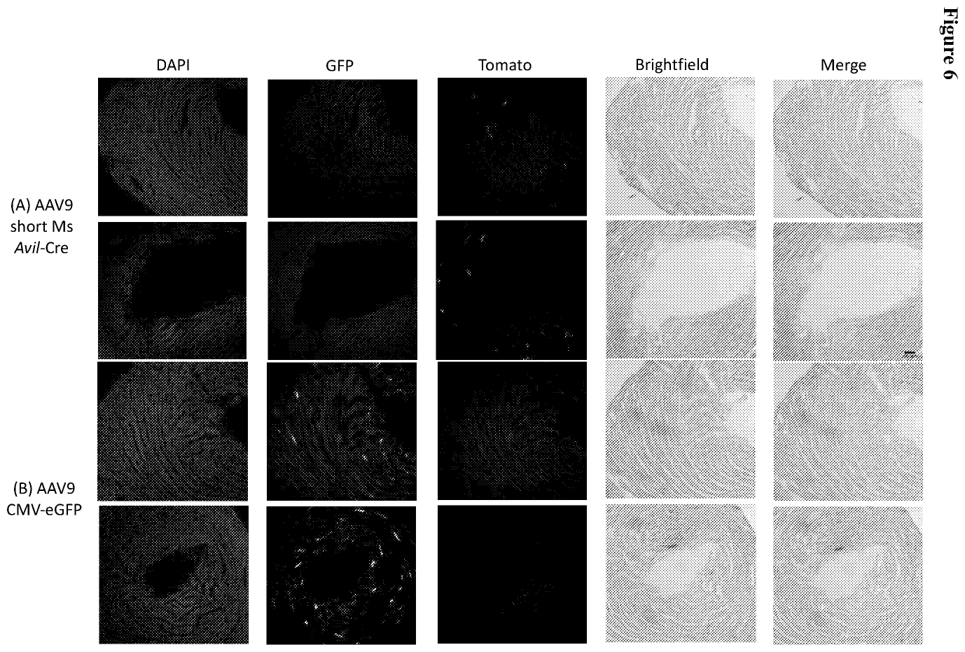

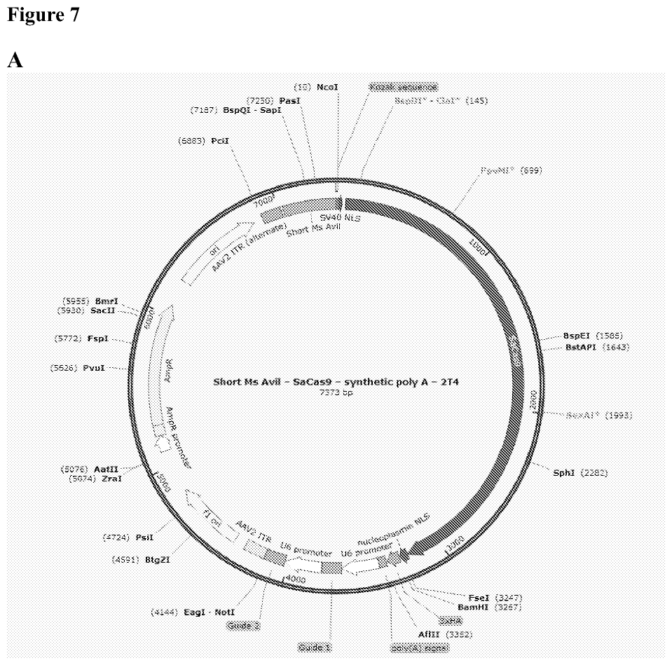

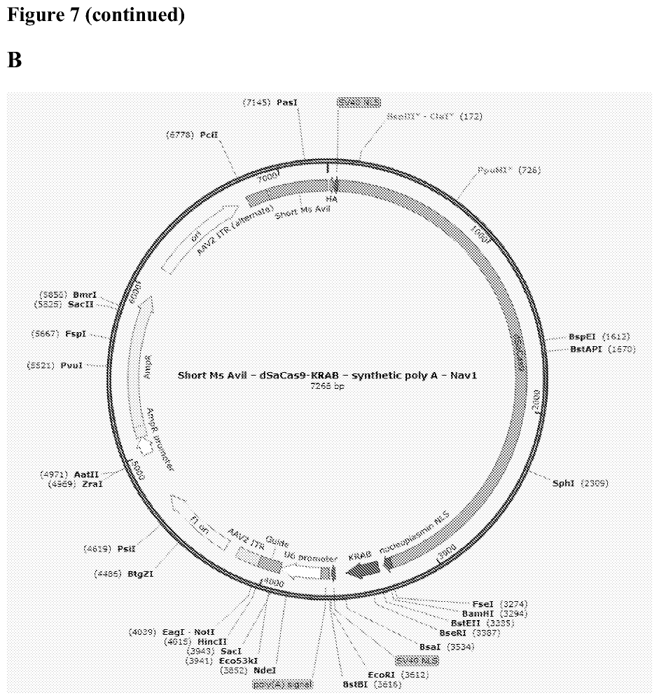

Plasmids 61591 (Ran et al. 2015) and 106213 (Thakore et al. 2018) (Addgene) were modified for the gene editing (SaCas9) and transcriptional repression (dSaCas9-KRAB) CRISPR experiments. The gene editing plasmid 61591 was first digested with AflII and NotI to remove the existing polyadenylation sequence and guide cassette. gBlocks gene fragments (IDT) were designed to contain a short synthetic polyadenylation sequence, U6 promoter, guide sequence and modified guide scaffold (Tabebordbar et al. 2016) with the design enabling two guide cassettes to be inserted into one plasmid by In-Fusion cloning (Takara). Guide sequences 1- CCACTTTATGTGGCTTGAAGG (SEQ ID NO: 10) and 2-AAGGAGAGAGCTGAGTCAGGT (SEQ ID NO: 11) were designed using the CRISPOR tool (Haeussler et al. 2016) and flank exon 1 of Scn9a. The modified plasmid was further digested with XbaI and AgeI to remove the CMV promoter which was replaced with the short mouse Avil promoter sequence (SEQ ID NO: 3) by In-Fusion cloning (Takara). The final plasmid is named Short Ms Avil-SaCas9-synthetic poly A-2T4 (FIG. 7A). Following SaCas9 nuclease activity at both sites, a microdeletion is induced that removes Scn9a promoter elements, transcriptional start site, exon 1 and flanking sequence.

For the transcriptional repression CRISPRi experiments the AgeI-EcoRI fragment from plasmid 106219 containing the dSaCas9-KRAB sequence was used to replace the SaCas9 sequence from plasmid 61591, to give a CMV driven dSaCas9-KRAB. Next, a gBlocks gene fragment (IDT) was designed to contain a synthetic poly(A) sequence, U6 promoter, guide sequence and modified guide scaffold (Tabebordbar et al. 2016). This sequence was cloned into the EcoRI-NotI sites of the modified plasmid 61591 using In-Fusion cloning (Takara). The guide sequence 3-TCCTCGATGCTCCCTGAGCTC (SEQ ID NO: 12) maps to exon 1 of Scn9a. The modified plasmid was further digested with XbaI and AgeI to remove the CMV promoter which was replaced with the short mouse Avil promoter sequence (SEQ ID NO: 3) by In-Fusion cloning (Takara). The final plasmid is named Short Ms Avil-dSaCas9-KRAB-synthetic poly A-Nav1 (FIG. 7B).

The SaCas9 and dSaCas9-KRAB plasmid cargoes targeting Scn9a were packaged into AAV1 (VectorBuilder) alongside a CMV-eGFP control AAV1 derived from plasmid VB150925-10026. The titres were 2.54×10¹³ GC/ml (SaCas9 2T4), 4.44×10¹³ GC/ml (dSaCas9-KRAB Nav1) and 1.49×10¹³ GC/ml (CMV eGFP).

### Example 5: Cancer-Induced Bone Pain Model

All experiments were performed in accordance with the UK Animals (Scientific Procedures) Act 1986 with prior approval under a Home Office project licence (PPL 70/7382). Mice were kept on a 12-h light/dark cycle and provided with food and water ad libitum. All animals were acclimatized for 2 weeks before the start of the experiment.

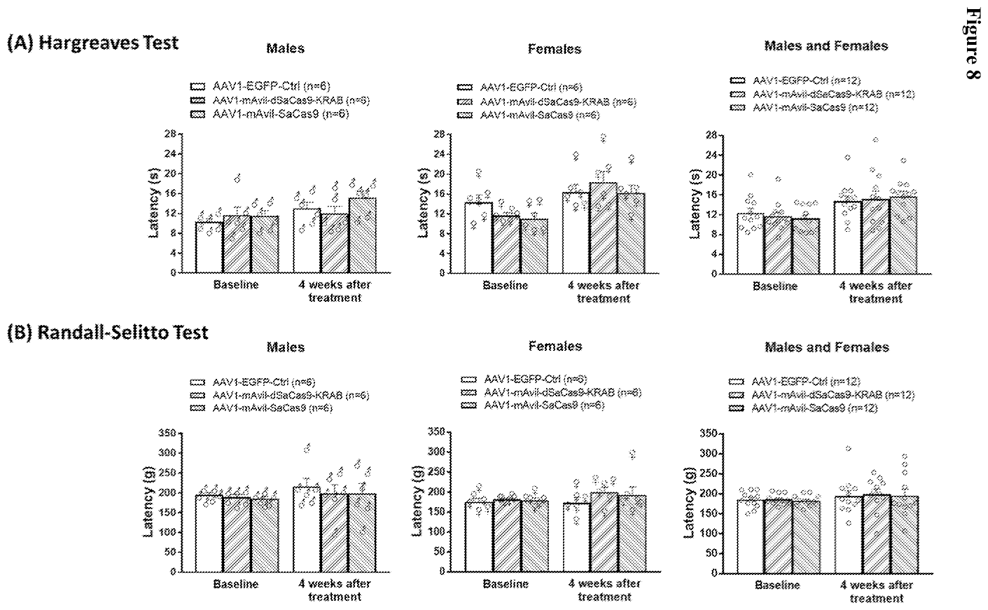

Three groups of 12 adult C57BL6/J mice (6 male and 6 female) were acclimatized to the behavioural equipment for at least 2 days prior to testing. Baseline values for rotarod (Stirling et al. 2005), Hargreaves' test (Hargreaves et al. 1988) (Minett et al. 2011), Randall Selitto (Randal and Selitto 1957) and von Frey tests (Minett et al. 2011) (Chaplan et al. 1994) (FIG. 8) were measured by two observers who were blind to the groups.

Following the baseline behaviour assays, the mice were injected intrathecally with the SaCas9, dSaCas9-KRAB or CMV eGFP AAV1 viruses. Briefly, mice were anesthetized with 2-3% isoflurane in 0.5 L/min oxygen following by shaving of the back region (from the insertion of the tail until the level of the ribs). The shaved region was then cleaned with ethanol 70% and mice restrained manually with a dorsal inclination that allowed the insertion of a 30-gauge needle attached to a cannula connected to a 10-μl microsyringe between the vertebras L5-L6 into the subdural space. Finally a volume of 5 μl of the AAV1 virus was given over a period of 5 seconds (Luiz et al. 2007) (Scheidt et al. 2002) (Gadotti et al. 2006). After the injections, the mice were observed till they had completely recovered from the anaesthesia. Four weeks later the Hargreaves test (FIG. 8A), Randall-Selitto test (FIG. 8B), von Frey test (FIG. 8C) and rota-rod test (FIG. 8D) were repeated.

Interestingly, there was little change to acute pain thresholds, potentially reflecting the fact that editing occurs only in a subset of all sensory neurons. This is valuable in terms of treating chronic pain whilst maintaining the ability to detect and respond to dangerous insults.

Mice were tested for their motor function on an accelerating Rotarod, and for innocuous sensation using von Frey filaments. Acute pain was measured using heat sensitivity with a Hargreaves apparatus. Mechanical pressure evoked pain was measured with a Randall Sellito apparatus. Four weeks after virus injection, the groups of mice were re-tested for motor function, innocuous sensation and response to noxious pressure and heat. There was little change to acute pain thresholds, potentially reflecting the fact that editing occurs only in a subset of all sensory neurons. This is valuable in terms of treating chronic pain whilst maintaining the ability to detect and respond to dangerous insults.

Thus, the promoters of the invention can be used to inhibit chronic pain pathways whilst maintaining the function of acute pain pathways.

### Cancer Cell Line

LL/2 Lewis Lung carcinoma cells were cultured in DMEM supplemented with 10% FBS and 1% Penicillin/Streptomycin for at least 2 weeks prior to surgery. Cells were split at 70-80% confluence and four days prior to surgery. On the day of surgery, cells were harvested with 0.05% Trypsin-EDTA and resuspended in DMEM at a final concentration of 2×10⁷ cells/ml and kept on ice till use. Viability of Lewis lung carcinoma cells was confirmed at the end of surgery, showing 26% dead cells compared to 11% before surgery.

### Bone Cancer Surgery

The bone cancer was introduced as previously described (Minett et al. 2014) six weeks after initial intrathecal delivery of AAV1 viruses. Briefly, animals were anaesthetised with isoflurane and sterile Lacri-Lube applied to their eyes. The surgical procedure was carried out under aseptic conditions. An incision was made in the skin lateral to the left patella. The lateral site of the patella tendon and lateral retinaculum tendon were loosened and the patella pushed aside to expose the distal femoral epiphysis (Falk et al. 2013). A 30-gauge needle was used to drill a hole into the medullary cavity through which 2×10⁵ Lewis lung carcinoma cells in 10 μl DMEM medium were inoculated with a 0.3 ml insulin syringe. The hole was closed with bone wax (Johnson & Johnson) and the wound thoroughly irrigated with sterile saline. The patella tendon was put back in place. The skin was sutured with 6-0 absorbable vicryl rapid (Ethicon), and Lidocaine spray (Intubeaze, 20 mg/ml, Dechra) applied to the wound. Mice were monitored daily after surgery and mice excluded from behaviour tests if the surgery was unsuccessful.

### Bone Cancer Behaviour

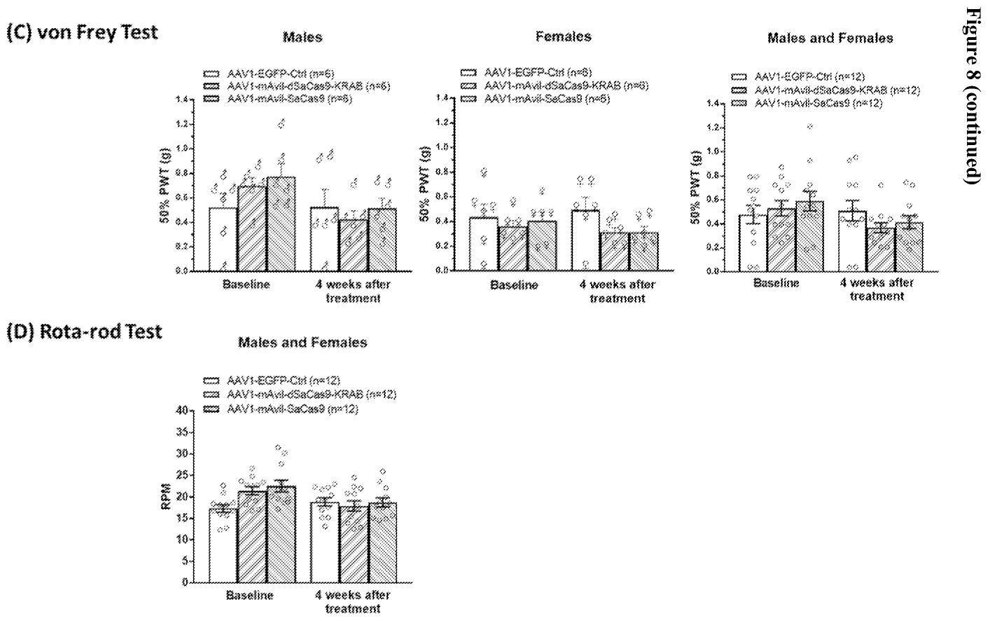

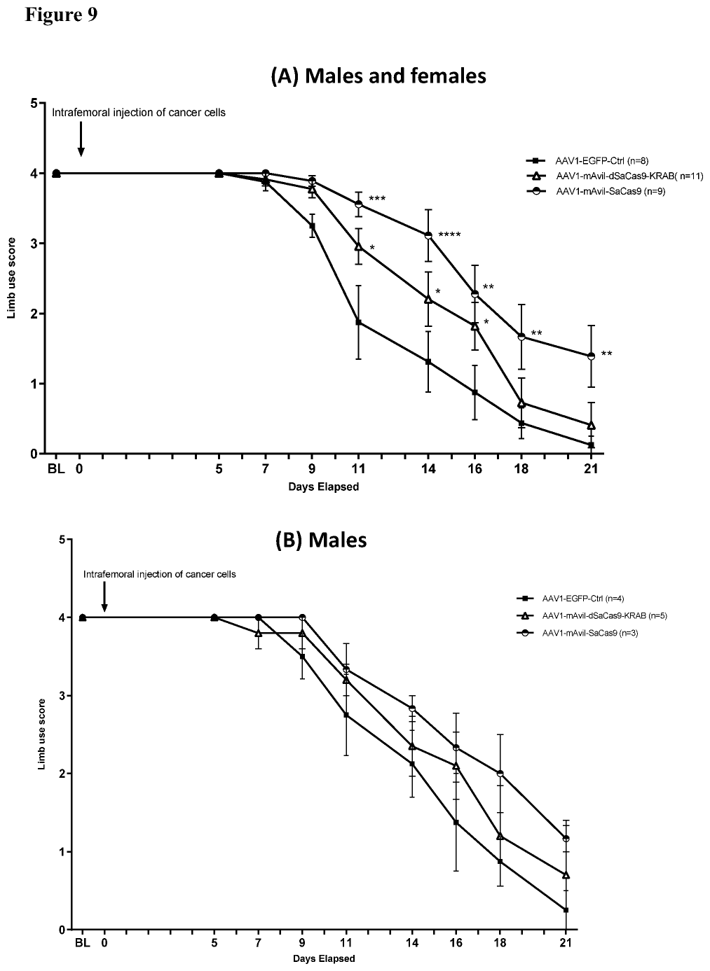

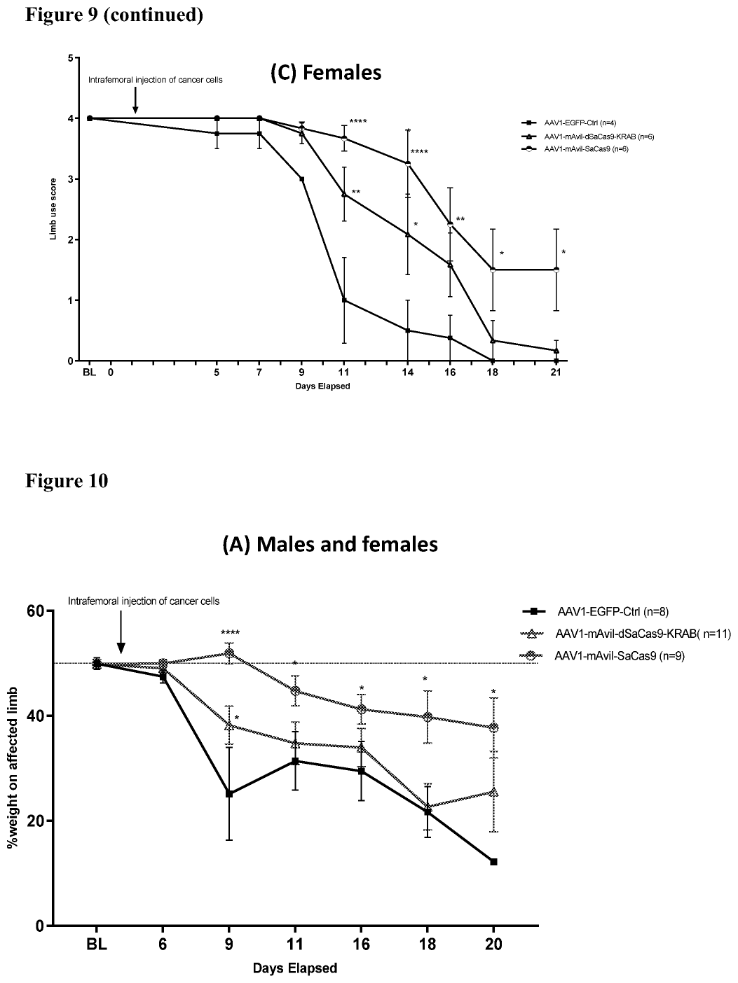

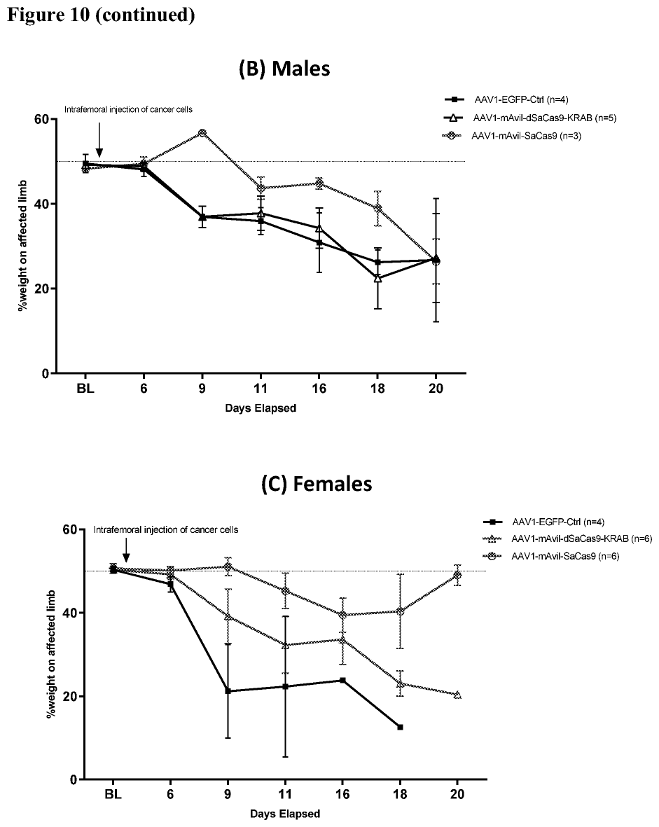

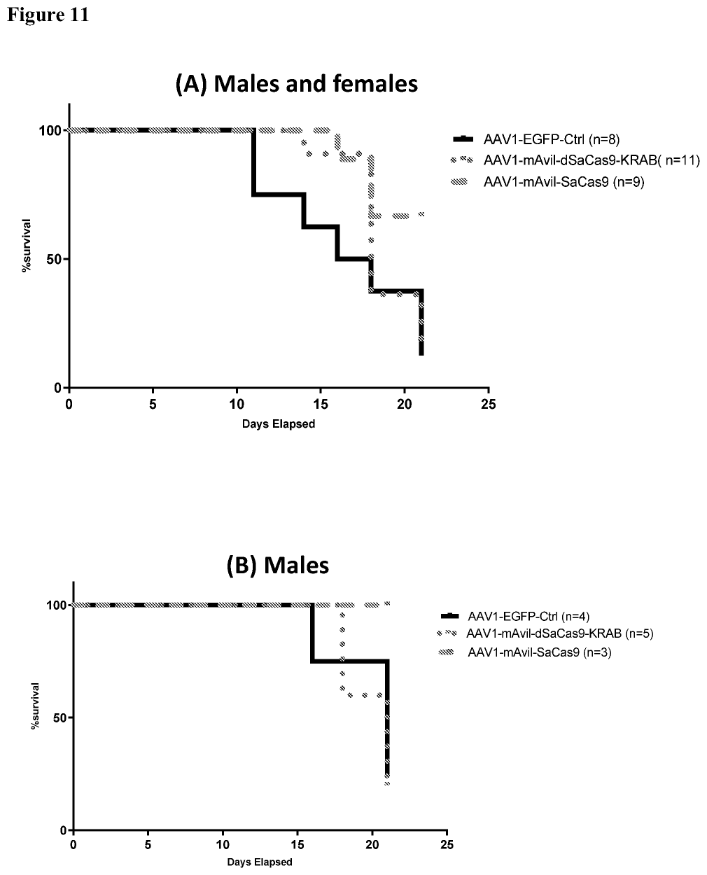

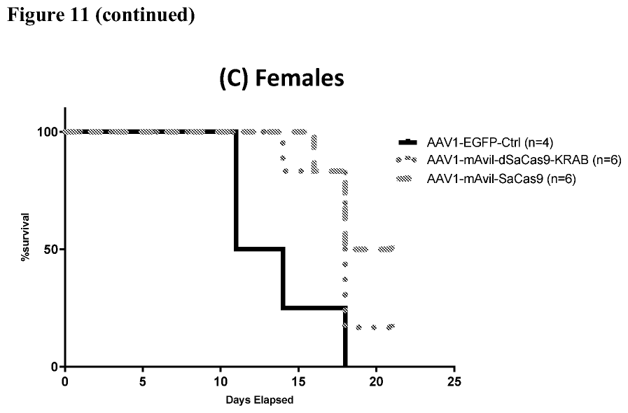

Limb use score: Mice were allowed to freely move around in a glass box. After 5-10 min of acclimatization, each mouse was observed for a period of 5 min and the use of the affected limb was scored as follows. 4: Normal use of hind limb, 3: insignificant limping, 2: significant limping, 1: significant limping and partial lack of limb use and 0: total lack of limb use.
Weight Bearing: Changes in weight-bearing were measured using an incapacitance meter consisting of two scales. The mouse was allowed to place its head and upper body into a plastic tube to reduce stress and the hind limbs were positioned each on one of the scales. The load of each limb on the scale was measured for 5 seconds in which the mouse was still. Measurements were taken in triplicate, changing the position of the hind legs after each trial. The average weight-bearing ratio was calculated as the weight placed on the affected limb divided by the total weight on both hind limbs. Statistical differences were determined using a two-way analysis of variance.
For chronic cancer pain, use of the affected limb was used to score pain (FIG. 9), as well as weight bearing (FIG. 10) and survival rates (FIG. 11) were also assessed. Results demonstrated a statistically significant diminution of bone cancer pain in both male and female mice, although the effects were more pronounced in female mice. Interestingly, commensurate with the diminished pain, there was an extension of lifetime with the female mice that was more pronounced than with male mice.

### REFERENCES

Altschul S. F. A protein alignment scoring system sensitive at all evolutionary distances J. Mol. Evol. 36, 290-300 (1993).
Altschul, S. F. et al., Basic local alignment search tool J. Mol. Biol., 215, 403-410 (1990).
Ayuso E. et al. High AAV vector purity results in serotype- and tissue-independent enhancement of transduction efficiency. Gene Ther. 17, 503-510 (2010).
Chaplan, S. R., Bach, F. W., Pogrel, J. W., Chung, J. M. & Yaksh, T. L. Quantitative assessment of tactile allodynia in the rat paw. J Neurosci Methods 53, 55-63 (1994).
Choi, J. S. et al. Paroxysmal extreme pain disorder: a molecular lesion of peripheral neurons. Nat Rev Neurol 7, 51-5 (2011).
Coura Rdos S. and Nardi, N. B., The state of the art of adeno-associated virus-based vectors in gene therapy, Virol. 1, 4, 99 (2007).
Devereux, J. et al. A comprehensive set of sequence analysis programmes for the VAX. Nucl. Acids Res. 12, 387-395 (1984).
Dib-Hajj, S. D. & Waxman, S. G. Sodium Channels in Human Pain Disorders: Genetics and Pharmacogenomics. Annu Rev Neurosci 42, 87-106 (2019).
Dong, J. Y. et al., Quantitative analysis of the packaging capacity of recombinant adeno-associated virus, Hum. Gene Ther., 7, 2101-2112 (1996).
Emery, E. C. et al. In vivo characterization of distinct modality-specific subsets of somatosensory neurons using GCaMP. Sci Adv 2, e1600990 (2016).
Elbashir et al., Nature, 411:494-498 (2001)
Elbashir et al., EMBO J., 20:6877-6888 (2001)
Erles, K., Sebokova, P. and Schlehofer, J. R., Update on the prevalence of serum antibodies (IgG and IgM) to adeno-associated virus (AAV), J Med. Virol. 59, 406-411 (1999).
Falk, S., Uldall, M., Appel, C., Ding, M. & Heegaard, A. M. Influence of sex differences on the progression of cancer-induced bone pain. Anticancer Res 33, 1963-9 (2013).
Gadotti, V. M. et al. Contribution of spinal glutamatergic receptors to the antinociception caused by agmatine in mice. Brain Res 1093, 116-22 (2006).
Haeussler, M. et al. Evaluation of off-target and on-target scoring algorithms and integration into the guide RNA selection tool CRISPOR. Genome Biol 17, 148 (2016).
Hargreaves, K., Dubner, R., Brown, F., Flores, C. & Joris, J. A new and sensitive method for measuring thermal nociception in cutaneous hyperalgesia. Pain 32, 77-88 (1988).
Henikoff, S. and Henikoff, J. G., Proc. Natl. Acad. Sci. USA 89, 10915-10919 (1992).
Hermonat, P. L. et al., Genetics of adeno-associated virus: isolation and preliminary characterization of adeno-associated virus type 2 mutants, J. Virol., 51, 329-339 (1984).
Joung and Sander. TALENs: a widely applicable technology for targeted genome editing. Nat Rev Mol Cell Biol. 1: 49-55.
Karlin, S. and Altschul, S. F., Applications and statistics for multiple high-scoring segments in molecular sequences Proc. Natl. Acad. Sci. USA 90: 5873-5787 (1993).
Konermann et al., Genome-scale transcriptional activation by an engineered CRISPR-Cas9 complex, Nature, Vol 000 (2014).
Kotin, R. M. et al., Site-specific integration by adeno-associated virus, Proc. Natl. Acad. Sci U S. A 87, 2211-2215 (1990).
Kuscue C., et al., CRISPR-STOP: gene silencing through base-editing-induced nonsense mutations, Nature, 14, 710-712 (2017).
Luiz, A. P. et al. Antinociceptive action of ethanolic extract obtained from roots of Humirianthera ampla Miers. J Ethnopharmacol 114, 355-63 (2007).
Magnus C. J. et al., Ultrapotent chemogenetics for research and potential clinical applications. Science, 364, eaav5282 (2019).
Manno, C. S. et al., Successful transduction of liver in hemophilia by AAV-Factor IX and limitations imposed by the host immune response, Nat. Med. (2006).
Michelfelder, S. and Trepel, M., Adeno-associated viral vectors and their redirection to cell-type specific receptors, Adv. Genet., 67, 29-60 (2009).
Minett, M. S., Quick, K. & Wood, J. N. Behavioral Measures of Pain Thresholds. Curr Protoc Mouse Biol 1, 383-412 (2011).
Minett, M. S. et al. Pain without nociceptors? Nav1.7-independent pain mechanisms. Cell Rep 6, 301-12 (2014).
Moreno A. M et al. Long-lasting Analgesia via Targeted in vivo Epigenetic Repression of Nav1.7. bioRxiv Jul. 24, 2019; doi: http://dx.doi.org/10.1101/711812.
Moskalenko, M. et al., Epitope mapping of human anti-adeno-associated virus type 2 neutralizing antibodies: implications for gene therapy and virus structure, J. Virol., 74, 1761-1766 (2000).
Ran, F. A. et al. In vivo genome editing using Staphylococcus aureus Cas9. Nature 520, 186-91 (2015).
Randall, L. O. & Selitto, J. J. A method for measurement of analgesic activity on inflamed tissue. Arch Int Pharmacodyn Ther 111, 409-19 (1957).
Reynolds et al., Nature Biotech., 22(3):326-330 (2004).
Scaringe et al., Nucl. Acids Res., 18:5433 (1990).
Scheidt, C. et al. Evidence for the involvement of glutamatergic receptors in the antinociception caused in mice by the sesquiterpene drimanial. Neuropharmacology 43, 340-7 (2002).
Stirling, L. C. et al. Nociceptor-specific gene deletion using heterozygous NaV1.8-Cre recombinase mice. Pain 113, 27-36 (2005).
Summerford, C. and Samulski, R. J., Membrane-associated heparan sulfate proteoglycan is a receptor for adeno-associated virus type 2 virions, J. Virol., 72, 1438-1445 (1998).
Sun, J. Y. et al., Immune responses to adeno-associated virus and its recombinant vectors, 10, 964-976 (2003).
Tabebordbar, M. et al. In vivo gene editing in dystrophic mouse muscle and muscle stem cells. Science 351, 407-411 (2016).
Thakore, P. I. et al. RNA-guided transcriptional silencing in vivo with S. aureus CRISPR-Cas9 repressors. Nat Commun 9, 1674 (2018).
Thomas, C. E. et al., Rapid uncoating of vector genomes is the key to efficient liver transduction with pseudotyped adeno-associated virus vectors, J. Virol. 78, 3110-3122 (2004).
Usman et al., J. Am. Chem. Soc., 109:7845 (1987).
Vincent-Lacaze, N. et al., Structure of adeno-associated virus vector DNA following transduction of the skeletal muscle, J Virol. 73, 1949-1955 (1999).
Wincott et al., Nucl. Acids Res., 23:2677-2684 (1995).
Wincott et al., Methods Mol. Bio., 74:59 (1997).
Wu, Z. et al., Adeno-associated virus serotypes: vector toolkit for human gene therapy, Mol. Ther., 14, 316-327 (2006).
Zaiss, A. K. and Muruve, D. A., Immunity to adeno-associated virus vectors in animals and humans: a continued challenge. Gene Ther 15, 808-816 (2008).

1. An expression construct comprising in a 5′ to 3′ direction:
(a) (i) an Advillin (Avil) promoter consisting of:
(1) a sequence of no more than 500 contiguous nucleotides comprising SEQ ID NO:1, or
(2) a sequence of no more than 500 contiguous nucleotides having 90% sequence identity to SEQ ID NO: 1, that retains the ability to express a payload sequence in dorsal root ganglion (DRG) neurons or the trigeminal ganglia; and
(b) a payload sequence,
wherein the promoter of (a) is operably linked to the payload sequence of (b).

2. The expression construct according to claim 1, wherein the Avil promoter consists of no more than 450 contiguous nucleotides and comprises:
(a) SEQ ID NO:1, or
(b) a sequence having 90% sequence identity to SEQ ID NO: 1, that retains the ability to express a payload sequence in DRG neurons or the trigeminal ganglia.

3. The expression construct according to claim 2, wherein the Avil promoter consists of no more than 400 contiguous nucleotides and comprises:
(a) SEQ ID NO:1, or
(b) a sequence having 90% sequence identity to SEQ ID NO: 1, that retains the ability to express a payload sequence in DRG neurons or the trigeminal ganglia.

4. The expression construct according to claim 1, wherein the Avil promoter consists of no more than 500 contiguous nucleotides and comprises SEQ ID NO:2, SEQ ID NO: 3, SEQ ID NO: 4, or SEQ ID NO: 5.

5. The expression construct according to claim 2, wherein the Avil promoter consists of no more than 450 contiguous nucleotides and comprises SEQ ID NO:2, SEQ ID NO: 3, SEQ ID NO: 4, or SEQ ID NO: 5.

6. The expression construct according to claim 3, wherein the Avil promoter consists of no more than 400 contiguous nucleotides and comprises SEQ ID NO: 2, SEQ ID NO: 3, SEQ ID NO: 4, or SEQ ID NO: 5.

7. The expression construct according to claim 6, wherein the Avil promoter consists of SEQ ID NO: 2, SEQ ID NO: 3, SEQ ID NO: 4, or SEQ ID NO: 5.

8. The expression construct according to any of the preceding claims, wherein the payload sequence comprises a transgene such as a KCNS1 gene, a SaCas9, a dSaCas9-KRAB, a dSaCas9-VPR, a double-stranded RNA, a mRNA, a ncRNA, a shRNA, siRNA, a miRNA, a guide RNA, a zinc-finger protein (ZFP), a transcription activator-like effector nuclease (TALEN) or a DREADD.

9. A vector comprising the expression construct according to any one of claims 1 to 8.

10. The vector according to claim 9, which is a viral vector.

11. The viral vector according to claim 10, which is an adeno-associated virus (AAV) vector or comprises an AAV genome or a derivative thereof.

12. The AAV vector according to claim 11, wherein said derivative is a chimeric, shuffled or capsid modified derivative.

13. The AAV vector according to claim 11 or 12, wherein said AAV genome is from a naturally derived serotype or isolate or clade of AAV.

14. The AAV vector according to claim 13, wherein said AAV genome is from AAV serotype 2 (AAV2), AAV serotype 4 (AAV4), AAV serotype 5 (AAV5) or AAV serotype 8 (AAV8) and/or wherein the capsid is derived from AAV1 or AAV9.

15. The AAV vector according to claim 14, wherein the genome is derived from AAV2 and the capsid is derived from AAV1 or AAV9.

16. The vector according to claim 9 or 10 or the AAV vector according to any of claims 11 to 15, further comprising a sequence encoding one or more guide RNAs if the payload sequence comprises SaCas9, dSaCas9-KRAB or dSaCas9-VPR.

17. The vector or AAV vector according to claim 16, wherein the one or more guide RNAs are directed to a Nav1.7 gene or Cav2.2 gene.

18. The vector according to any one of claim 9, 16 or 17, which is packaged in a vesicle, liposome, exosome or nanoparticle.

19. A host cell that produces the vector of any one of claim 9, 10, 16 or 17, or the AAV vector of any one of claims 11 to 17.

20. A pharmaceutical composition comprising the vector of any one of claim 9, 10, 16, 17 or 18, or the AAV vector of any one of claims 11 to 18, and a pharmaceutically acceptable carrier.

21. The vector according to any one of claim 9, 10, 16, 17 or 18, or the AAV vector according to any one of claims 11 to 18, or the pharmaceutical composition of claim 20, for use in a method of treating or preventing chronic pain.

22. Use of the vector according to any one of claim 9, 10, 16, 17 or 18, or the AAV vector according to any one of claims 11 to 18, or the pharmaceutical composition of claim 20, in the manufacture of a medicament for the treatment or prevention of chronic pain.

23. A method of treating to preventing chronic pain in a patient in need thereof, comprising administering a therapeutically effective amount of the vector according to any one of claim 9, 10, 16, 17 or 18, or the AAV vector according to any one of claims 11 to 18, or the pharmaceutical composition of claim 20, to said patient.

24. The vector or AAV vector or pharmaceutical composition for use according to claim 21, the use of claim 22, or the method according to claim 23, wherein the chronic pain is cancer pain, cancer-associated bone pain, or chronic pain associated with rheumatoid arthritis, osteoarthritis, trigeminal neuralgia, headache, migraine, fibromyalgia, diabetic neuropathy, or other neuropathy associated pain, neuropathic pain, idiopathic pain, erythromelalgia and/or paroxysmal extreme pain disorder.

25. The vector or AAV vector or pharmaceutical composition for use according to claim 21 or 24, the use of claim 22 or 24, or the method according to claim 23 or 24, wherein the vector or AAV vector or pharmaceutical composition is administered parentally, preferably intravenously or intrathecally, to a patient.


SEQUENCE LISTING (19 sequences)

SEQ ID NO: 1 - DNA, 394 bp, Artificial Sequence
  Advillin consensus sequence
  misc_feature (1)..(1): Position 1 is C or no nucleotide
  misc_feature (2)..(2): Position 2 is C or no nucleotide
  misc_feature (3)..(3): Position 3 is G, A or no nucleotide
  misc_feature (4)..(4): Position 4 is G or no nucleotide
  misc_feature (5)..(5): Position 5 is C or no nucleotide
  misc_feature (6)..(6): Position 6 is C or no nucleotide
  misc_feature (57)..(57): Position 57 is T or no nucleotide
  misc_feature (73)..(73): Position 73 is C or no nucleotide
  misc_feature (74)..(74): Position 74 is T or no nucleotide
  misc_feature (75)..(75): Position 75 is C or no nucleotide
  misc_feature (76)..(76): Position 76 is T or no nucleotide
  misc_feature (77)..(77): Position 77 is T or no nucleotide
  misc_feature (78)..(78): Position 78 is T or no nucleotide
  misc_feature (79)..(79): Position 79 is C or no nucleotide
  misc_feature (80)..(80): Position 80 is C or no nucleotide
  misc_feature (100)..(100): Position 100 is T or no nucleotide
  misc_feature (101)..(101): Position 101 is G or no nucleotide
  misc_feature (121)..(121): Position 121 is A or no nucleotide
  misc_feature (150)..(150): Position 150 is A or no nucleotide
  misc_feature (151)..(151): Position 151 is A or no nucleotide
  misc_feature (152)..(152): Position 152 is G or no nucleotide
  misc_feature (153)..(153): Position 153 is A or no nucleotide
  misc_feature (154)..(154): Position 154 is A or no nucleotide
  misc_feature (155)..(155): Position 155 is A or no nucleotide
  misc_feature (156)..(156): Position 156 is A or no nucleotide
  misc_feature (184)..(184): Position 184 is T or no nucleotide
  misc_feature (188)..(188): Position 188 is T or no nucleotide
  misc_feature (222)..(222): Position 222 is C or no nucleotide
  misc_feature (223)..(223): Position 223 is C, T or no nucleotide
  misc_feature (330)..(330): Position 330 is G or no nucleotide
  misc_feature (331)..(331): Position 331 is C or no nucleotide
  misc_feature (332)..(332): Position 332 is T or no nucleotide
  misc_feature (333)..(333): Position 333 is G or no nucleotide
  misc_feature (334)..(334): Position 334 is T or no nucleotide
  misc_feature (351)..(351): Position 351 is T or no nucleotide
  misc_feature (371)..(371): Position 371 is C or no nucleotide
  misc_feature (372)..(372): Position 372 is C or no nucleotide
  misc_feature (373)..(373): Position 373 is T or no nucleotide
  misc_feature (374)..(374): Position 374 is C or no nucleotide
  misc_feature (375)..(375): Position 375 is C or no nucleotide
  misc_feature (376)..(376): Position 376 is C, T or no nucleotide
  misc_feature (377)..(377): Position 377 is G or no nucleotide
  misc_feature (378)..(378): Position 378 is C or no nucleotide
  misc_feature (379)..(379): Position 379 is C, T or no nucleotide
  misc_feature (380)..(380): Position 380 is C, T or no nucleotide
  misc_feature (381)..(381): Position 381 is C, T or no nucleotide
  misc_feature (382)..(382): Position 382 is G, T or no nucleotide
  misc_feature (383)..(383): Position 383 is G or no nucleotide
  misc_feature (384)..(384): Position 384 is A, G or no nucleotide
  misc_feature (385)..(385): Position 385 is G or no nucleotide
  misc_feature (386)..(386): Position 386 is T or no nucleotide
  misc_feature (387)..(387): Position 387 is G or no nucleotide
  misc_feature (388)..(388): Position 388 is G or no nucleotide
  misc_feature (389)..(389): Position 389 is T or no nucleotide
  misc_feature (390)..(390): Position 390 is T or no nucleotide
  misc_feature (391)..(391): Position 391 is C or no nucleotide
  misc_feature (392)..(392): Position 392 is C, T or no nucleotide
  misc_feature (393)..(393): Position 393 is C, T or no nucleotide
  misc_feature (394)..(394): Position 394 is C, T or no nucleotide
nnnnnnycwkyctcctgtcatcasyrtaatryrtyyayatctmcaatatggcaaatntcatatcctyccwmmnnnnnnnnctgmattattgatggvcwrnntgcacttttwaaaaaatcanwtagaycagggygtggarctggartymannnnnnnrrgargcctttaraagtcygcwcttctngttnyygctgttttgmataggmrcagataaagcttycnnctctggtttgaataagycaagcycagkgctaggttggcystgattggycagvrctrrgaraatkhggttawgatgcaaacmcaagcaaatataacccdgwrtctvwnnnnngcrgccattrctrarmnmaggcwkcaggasskgvhgnnnnnnnnnnnnnnnnnnnnnnnn

SEQ ID NO: 2 - DNA, 378 bp, Artificial Sequence
  human Advillin promoter fragment
ccagcctcttcctcctgtcatcaccataatgcatccatatctacaatatggcaaatttcatatccttccaacctctttcctgcattattgatgggctgtgtgcactttttaaaaaatcaattagatcagggcgtggagctggagttcaaagaagcctttaaaagtctgctcttctgttttgctgttttgaataggcacagataaagctttccctctggtttgaataagccaagctcagtgctaggttggctctgattggccaggactaggaaaatgcggttaagatgcaaacacaagcaaatataacccagtatctctgcggccattactaagctaaggcagcaggacctggagcctcctgctttggagtggttcttc

SEQ ID NO: 3 - DNA, 344 bp, Artificial Sequence
  mouse Advillin promoter fragment
ccagtctcctgtcatcagcataatacgttcatatctacaatatggcaaattcatatcctccctcactgaattattgatggacagtgcacttttaaaaaaatcaatagaccagggtgtggagctggagttaaaagaagcctttagaagtctgctcttcttgttttcgctgttttgcataggagcagataaagctttcctctctggtttgaataagtcaagcccagggctaggttggctgtgattggccagagctaggagaatgtggttatgatgcaaacacaagcaaatataaccctgtgtctatgctgtgcggccattgctgaaacaggcttcaggacgggctg

SEQ ID NO: 4 - DNA, 376 bp, Artificial Sequence
  pig Advillin promoter fragment
ccagcccctgcctcctgtcatcagtgtaatgtgtctacatctccaatatggcaaatttcatatcctcccaccctctttctgcattattgatggcctgtgcacttttaaaaaaatcaattagatcagggcgtggagctggagttaaaagaagcctttaaaagtctgcacttcttgttttgctgttttgaataggaacagataaagctttccctctggtttgaataagtcaagcccagggctaggttggctgtgattggtcaggactaagaaaatgcggttaagatgcaaacccaagcaaatataacccagtgtctgagcagccattactgagctaaggcagcaggaccggaagcctcccgcttttgggtggttcctc

SEQ ID NO: 5 - DNA, 386 bp, Artificial Sequence
  dog Advillin promoter fragment
ccggcccctgcctcctgtcatcagcataatgcatccatatctacaatatggcaaatttcatatcctcccaccctctttccctgcattattgatgggctatgcacttttaaaaaaatcaattagatcagggtgtggaactggaatcaaaagaaaagggaggcctttaaaagtccgctcttcttgtttctgctgttttgaataggagcagataaagcttcccctctggtttgaataagtcaagcccagggctaggttggccgtgattggccagcactgggaaaattaggttaagatgcaaacacaagcaaatataacccggagtctgtgcagccattgctaagctaaggcagcaggagctggcgcctcccgccccggagtggttctct

SEQ ID NO: 6 - DNA, 1298 bp, Artificial Sequence
  human Advillin larger promoter region (including exon 1)
tgtgagctgccaccaaccttcattggcccttaagtgttcactgggcccatggcaagcaccgcctttcttctcgcaaccccttccatcatgaagcagctgcacccccgggctgagtggggtgttgcacccaccgggaggccagctgcagatgccacacagggcttccgctcaaggttacacaagttctgctgtgatcaaccccattgcagtcaggtgcttagcacgtgtatatgcttgatgtcctattgacttctgggcatgaaccagaagccctaagggaatggattattatttttttttttttttttgaaatggagtcttgctctgttgcccaggctagaggcagtggcatgatctcagctcactgcaacctccacctcccaggttcaagcgattctcctgcctcagtctcctgagtagctgggattacaggcgcctgccagcacgcccggctcatttttgtatttttagtagagatggggtttcaccatcttggccaggctgttttcaaactcctcacattgtgatccacccacctcagcctcccaaagtgctgggattacaggcgtgagccaccaccacacccagccctgggaatggagtcttgattcttctctgcccctcactgattcttccaaactggaaaccctgaagctgagagcccagcatggttcctggcaaacagcaggcactcaaatattgattggtttactgtatgactagtagagaccccaacgagcaaaactgtggcctaataaaattctggctcctctcccagacttcccctccctttgagaaatgccagaagcttcttagggaggctcttgccaacctagacatcacaggcactcatggggcagctccagcctcttcctcctgtcatcaccataatgcatccatatctacaatatggcaaatttcatatccttccaacctctttcctgcattattgatgggctgtgtgcactttttaaaaaatcaattagatcagggcgtggagctggagttcaaagaagcctttaaaagtctgctcttctgttttgctgttttgaataggcacagataaagctttccctctggtttgaataagccaagctcagtgctaggttggctctgattggccaggactaggaaaatgcggttaagatgcaaacacaagcaaatataacccagtatctctgcggccattactaagctaaggcagcaggacctggagcctcctgctttggagtggttcttcaatactgctgctgcttacgcgccgggaaactgggaaggctggtgagcgagaaggcaag

SEQ ID NO: 7 - DNA, 856 bp, Artificial Sequence
  mouse Advillin larger promoter region (including exon 1)
actgggagctgcttttgctttccttcccttccactgacctgacactcggtggacactgttcttcctgcatgacctcgttgatcgtgaaatgatagcacctggcagagcaaacgctgggattctgatcaaggttacagaggttggagtgcattagcccctctcgggcctgggttggaaccaactccctctccccaggtacttggcaagcgtttgcttgctgtgctattgactggggtagagactgaagccctgggggaattggaggctcctgcatgtgctcttgatgctgccaaacaggccttgagattcagagcccagcatggtttccaggcacatagtaggatgacttgtacagactccaagcaacaaaagctttcctgacaccacagaattgcccagaatcatctctcagccctgaacgtccaggcacacagtaggggagctctagccagtctcctgtcatcagcataatacgttcatatctacaatatggcaaattcatatcctccctcactgaattattgatggacagtgcacttttaaaaaaatcaatagaccagggtgtggagctggagttaaaagaagcctttagaagtctgctcttcttgttttcgctgttttgcataggagcagataaagctttcctctctggtttgaataagtcaagcccagggctaggttggctgtgattggccagagctaggagaatgtggttatgatgcaaacacaagcaaatataaccctgtgtctatgctgtgcggccattgctgaaacaggcttcaggacgggctgctttgggagacctcatcagtacttcacctgctgctttgccggacttcggaggacagagggtcag

SEQ ID NO: 8 - DNA, 937 bp, Artificial Sequence
  pig Advillin larger promoter region (including exon 1)
atcctgctgttgttcatttgggcacttaaccccttcttccctcaaaagccctctgtggtgatgaatgatgaagccagagcgcccaggctgggaggccagctacggactcagcacaggggttgtgctccaggttacagaagtctggctatgttagctcttccctggcctccatgtggaaccaagcccagtgccatccaggtgcttaataagcatatgcttggggtcttaactgacttctagggaggaaccagaagccccaagggacttggactcttctttcttctgtgctcctcactgattttcccaaactaggagccctgaaactcagcccagggtagttcctggtacttagtaggcactgagatactgattggatggttttgcccaaggggcaaaacaggatgatgtaataacatactcctggcccctctcccagactgcccctccgagaaatgcccagctgggaggggcttgccagcctggacatcccaggtgctcagggggcagccccagcccctgcctcctgtcatcagtgtaatgtgtctacatctccaatatggcaaatttcatatcctcccaccctctttctgcattattgatggcctgtgcacttttaaaaaaatcaattagatcagggcgtggagctggagttaaaagaagcctttaaaagtctgcacttcttgttttgctgttttgaataggaacagataaagctttccctctggtttgaataagtcaagcccagggctaggttggctgtgattggtcaggactaagaaaatgcggttaagatgcaaacccaagcaaatataacccagtgtctgagcagccattactgagctaaggcagcaggaccggaagcctcccgcttttgggtggttcctcaacactaattatccgccaaaaaactgggaagactggtgagagagaaaacaag

SEQ ID NO: 9 - DNA, 974 bp, Artificial Sequence
  dog Advillin larger promoter region (including exon 1)
ccctggtgtcgttcattcgggagtcaccggcacactcttcttcccgtgcatccaaccccttgtgtcacggcagagctgcgcacccaggctgggcaggctgttgctccggccgggaggccagctacggactcggctcggcttccgctcaaggttacagaagttgcactacatgagttctgttctggccccccgtgtggaagcagctccgttgccgtccaggtgcttgataagcatacgcttggtgtcccattgacttctgggaatgaaccaggagccccgagggagtaggagtcttctgtgccccttactgcttctcccagactgggatccccggaacctagaatccatgattcttggcacatagtaggcactcagattctggttggactgttgtatggaccccaagggaagccctaaagagcaaaacaggatgacctaatataacacatcctggcccctcccctagacctctctgcctctgagaaatacccagaagcttctcagggaggcacttgccagcccaggcactcaggggcagctccggcccctgcctcctgtcatcagcataatgcatccatatctacaatatggcaaatttcatatcctcccaccctctttccctgcattattgatgggctatgcacttttaaaaaaatcaattagatcagggtgtggaactggaatcaaaagaaaagggaggcctttaaaagtccgctcttcttgtttctgctgttttgaataggagcagataaagcttcccctctggtttgaataagtcaagcccagggctaggttggccgtgattggccagcactgggaaaattaggttaagatgcaaacacaagcaaatataacccggagtctgtgcagccattgctaagctaaggcagcaggagctggcgcctcccgccccggagtggttctctggataccgcctggaaacccggtgggagcccggccggagagcaggcaag

SEQ ID NO: 10 - DNA, 21 bp, Artificial Sequence
  Scn9a guide sequence 1
ccactttatgtggcttgaagg

SEQ ID NO: 11 - DNA, 21 bp, Artificial Sequence
  Scn9a guide sequence 2
aaggagagagctgagtcaggt

SEQ ID NO: 12 - DNA, 21 bp, Artificial Sequence
  Scn9a guide sequence 3
tcctcgatgctccctgagctc

SEQ ID NO: 13 - DNA, 3156 bp, Artificial Sequence
  SaCas9
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 14 - DNA, 3156 bp, Artificial Sequence
  dSaCas9
aagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 15 - DNA, 195 bp, Artificial Sequence
  KRAB domain
cggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtg

SEQ ID NO: 16 - DNA, 3345 bp, Artificial Sequence
  NLS-SaCas9-NLS-3xHA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaa

SEQ ID NO: 17 - DNA, 3600 bp, Artificial Sequence
  HA-NLS-dSaCas9-NLS-KRAB-NLS
atgtacccatacgatgttccagattacgctgccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtttaa

SEQ ID NO: 18 - DNA, 232 bp, Artificial Sequence
  Human EFS promoter sequence
ggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagg

SEQ ID NO: 19 - DNA, 145 bp, Artificial Sequence
  Mouse EFS promoter sequence
accggggagctggcgcggggcaaactgggaaagcggtgtcgtgtgctggctccgccctcttcccgagggtgggggagaacggtatataagtgcggcagtcgccttggacgttctttttcgcaacgggtttgccgtcagaacgcag